(19) United States
(12) Patent Application Publication
(10) Pub. No.: US 2005/0192281 A1
(43) Pub. Date: Sep. 1, 2005

(54) Nitrone compounds, prodrugs and pharmaceuticals compositons of the same to treat human disorders

(76) Inventors: Michael G. Kelly, Thousand Oaks, CA (US); Ravindra B. Upasani, San Jose, CA (US); Satyanarayana Janagani, Santa Clara, CA (US)

Correspondence Address: KLAUBER & JACKSON, 411 HACKENSACK AVENUE, HACKENSACK, NJ, 07601, US

(21) Appl. No.: 10/965,680

(22) Filed: Oct. 14, 2004

Related U.S. Application Data
(60) Provisional application No. 60/511,374, filed on Oct. 14, 2003.
(60) Provisional application No. 60/511,379, filed on Oct. 14, 2003.
(60) Provisional application No. 60/511,625, filed on Oct. 14, 2003.

(51) Int. Cl. A61K 31/517; A61K 31/502; A61K 31/44; A61K 31/47; A61K 31/13

(52) U.S. Cl. 514/248; 514/249; 514/266.4; 514/310; 514/357; 514/645; 514/311

(57) ABSTRACT

Disclosed are aryl, heteroaromatic and bicyclic aryl nitrone compounds and pharmaceutical compositions containing such derivatives. The disclosed compositions are useful for preventing and/or treating pain, neurodegenerative, autoimmune and inflammatory diseases or conditions in mammals.

### CROSS REFERENCE TO RELATED APPLICATIONS

The present application claims the priority of co-pending provisional applications U.S. Ser. Nos. 60/511,374, 60/511,379 and 60/511,625 filed on Oct. 14, 2003, and the disclosures of these application are incorporated by reference herein in their entireties. Applicants claim the benefits of these applications under 35 U.S.C. § 119(e).

### FIELD OF THE INVENTION

This invention relates to aryl, heteroaromatic and bicyclic aryl nitrone compounds and their use as therapeutic agents for the treatment of inflammation-related conditions in mammals such as (but not limited to) arthritis, neurodegenerative disorders such as (but not limited to) Parkinson's disease and Alzheimer's disease, stroke, uveitis, asthma, myocardial infarction, the treatment and prophylaxis of pain syndromes (acute and chronic or neuropathic), traumatic brain injury, acute spinal cord injury, alopecia (hair loss), inflammatory bowel disease and autoimmune disorders.

### BACKGROUND OF THE INVENTION

Arthritis and related inflammatory disease conditions occur in more than 100 different forms, including rheumatoid arthritis (RA), osteoarthritis (OA), ankylosing spondylitis and systemic lupus erythematosus (SLE). Most forms of arthritis are characterized by some type of chronic inflammation. For example, RA typically involves chronic inflammation of the lining of the joints and/or the internal organs. Such chronic inflammation generally causes pain and swelling in the joints of those afflicted and may result in damage to cartilage, bone, tendons, ligaments and the like, ultimately leading to deformity and disability.

Prostaglandins (PG) have long been known to be involved in the inflammation process. Accordingly, a number of inhibitors of PG synthesis have been developed for the treatment of arthritis and related inflammatory disease conditions. Such non-steroidal anti-inflammatory drugs (NSAIDs) typically prevent the production of PGs by inhibiting enzymes such as cycloxygenase (COX) and lipoxygenase. The enzyme COX is known to exist in two forms. COX-1 is a constitutive form found in most tissues and organs. Among other properties, COX-1 produces small amounts of PGs necessary for maintaining the integrity of the GI track. COX-2 is an inducible form associated with the increased production of PGs during inflammatory conditions. Since many NSAIDs inhibit both forms of COX, they interfere with PG-regulated processes not associated with the inflammation process. As a result, many NSAIDs cause severe side effects, such as stomach ulcers and renal damage, which limit their effectiveness as therapeutics.

Accordingly, a need exists for novel classes of therapeutic compounds which effectively treat arthritis and other inflammatory-related conditions without producing undesirable side effects.

Nitrones constitute a class of compounds that have antioxidant properties due to their ability to form stable adducts (i.e., spin traps) with free radicals. Since free radicals can cause oxidative damage to cellular constituents (e.g., proteins and lipids), which can lead to pathological consequences, it has been reported that the antioxidant properties of nitrones at least partly underlie their therapeutic potential. Therefore, diseases which have been reported to be susceptible to antioxidant therapy or which involve the generation of free radicals may be susceptible to nitrone treatment based on the antioxidant activity of nitrones.

Aromatic nitrone compounds such as C-(phenyl)-N-(tert-butyl)nitrone (PBN) and derivatives thereof have been reported as possible therapeutics for the treatment of a wide variety of disease conditions arising from or characterized by oxidative damage or oxidative stress. Nitrone compounds exhibiting improved antioxidant activity compared to PBN can have better therapeutic potential than PBN. Aromatic nitrone breakdown, metabolism or degradation products such as N-alkyl hydroxylamines, N-alkyl hydronitroxides or nitric oxide may also contribute to the antioxidant properties of the aromatic nitrones, and contribute to their interruption of the inflammatory signaling pathways. Disease conditions arising from or characterized by oxidative damage or stress include, for example, disorders of the CNS and the PNS, such as stroke, Parkinson's disease, nerve damage and the like, and disorders of the peripheral organs, such as atherosclerosis, cardiac infarction, ulcerative colitis and the like.

A need exists for new classes of aromatic nitrone derivatives that have improved properties such as low toxicity, increased solubility, improved cellular and blood-brain-barrier permeability, and improved oral bioavailability.

### SUMMARY OF THE INVENTION

Herein described are aromatic nitrone compounds that have improved antioxidant activity compared to PBN. The compounds of the invention are presented as potential therapeutic agents for indications that have been reported to be amenable to antioxidant treatment or that involve free-radical generation including, but not limited to: stroke, myocardial infarction and dysfunction, retinal ischemia and damage including macular degeneration and other degenerative disorders of the retina, renal ischemia, arteriosclerosis and other cardiovascular diseases, amyotrophic lateral sclerosis, Parkinson's disease, Alzheimer's disease, Huntington's disease, multiple sclerosis, head trauma and traumatic brain injury, nerve injury and neuropathies, migraine, schizophrenia and other disorders of cognition, mood disorders and other disorders of affect, pancreatitis and other pancreatic disorders, the treatment of diabetes and related complications, epilepsy, transplant and graft failure or rejection, hepatitis and jaundice-induced liver disorders, lung injury and damage, gastric ulcer, endotoxemia, aging and senescence, fetal damage due to intrauterine ischemia, the treatment and prophylaxis of pain syndromes (acute and chronic or neuropathic), arthritis and other autoimmune disorders, asthma and allergic reactions, inflammatory bowel disease, irritable bowel syndrome, uveitis, cancer, the treatment of complications and disorders arising from cancer therapy, and alopecia (hair loss).

The present invention provides aromatic nitrone compounds that are capable of modifying mammalian inflammatory pathways, pharmaceutical compositions having substituted aryl, heteroaromatic or bicyclic aryl nitrones as active ingredients and their use to treat, prevent or ameliorate a range of conditions in mammals such as, but not limited to, pain of various genesis or etiology, for example, acute, chronic, inflammatory and neuropathic pain, dental pain and headache (such as migraine, cluster headache and tension headache). The compounds of the present invention are also useful as anti-inflammatory agents for the treatment of arthritis, and as agents to treat Parkinson's disease, Alzheimer's disease, stroke, uveitis, asthma, myocardial infarction, traumatic brain injury, spinal cord injury, neurodegenerative disorders, alopecia (hair loss), inflammatory bowel disease, autoimmune disorders, renal disorders, obesity, eating disorders, cancer, schizophrenia, epilepsy, sleeping disorders, cognition, depression, anxiety, high blood pressure, lipid disorders and atherosclerosis.

In one aspect, the present invention provides aryl nitrone compounds that comprise a cycloalkenyl or aryl ring of 5 to 8 atoms. A first position of the ring is bonded to the carbon atom of a nitrone group via a linker L. The linker L can be a heteroalkyl chain. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to a group selected from substituted or unsubstituted alkyl, substituted or unsubstituted heteroalkyl, substituted or unsubstituted aryl, substituted or unsubstituted heteroaryl, substituted or unsubstituted aralkyl, or substituted or unsubstituted heteroaralkyl.

In a particular embodiment of the invention, the ring of the compound is a phenyl ring. The phenyl ring can be substituted only with the first group or the phenyl ring can be further substituted.

In a further aspect, the present invention provides pharmaceutical compositions comprising aryl nitrone compounds and a pharmaceutical carrier, excipient or diluent. In this aspect of the invention, the pharmaceutical composition can comprise an aryl nitrone compound described above.

In another aspect, the present invention provides heteroaromatic nitrone compounds that comprise a cycloheteroalkenyl or heteroaryl ring of 5 to 8 atoms. A first position of the ring is bonded to the carbon atom of a nitrone group via a linker L. The linker L can be alkyl or heteroalkyl chain. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to a group selected from substituted or unsubstituted alkyl, substituted or unsubstituted heteroalkyl, substituted or unsubstituted aryl, substituted or unsubstituted heteroaryl, substituted or unsubstituted aralkyl, or substituted or unsubstituted heteroaralkyl.

The ring can be substituted only with the first group or the ring can be further substituted.

In a further aspect, the present invention provides pharmaceutical compositions comprising heteroaromatic nitrone compounds and a pharmaceutical carrier, excipient or diluent. In this aspect of the invention, the pharmaceutical composition can comprise a heteroaromatic nitrone compound described above.

In yet another aspect, the present invention provides bicyclic aryl nitrone compounds that comprise a bicycloalkenyl, bicycloheteroalkenyl, bicycloaryl or bicycloheteroaryl ring of 8 to 11. A first position of the ring is bonded to the carbon atom of a nitrone group via a linker L. The linker L can be heteroalkyl chain. The carbon atom of the nitrone is further bonded to hydrogen, substituted or unsubstituted (C₁-C₆)alkyl, substituted or unsubstituted (C₁-C₆)cycloalkyl, substituted or unsubstituted aryl, or substituted or unsubstituted aralkyl. The nitrogen atom of the nitrone group is bonded to a group selected from substituted or unsubstituted alkyl, substituted or unsubstituted heteroalkyl, substituted or unsubstituted aryl, substituted or unsubstituted heteroaryl, substituted or unsubstituted aralkyl, or substituted or unsubstituted heteroaralkyl.

The ring can be substituted only with the first group or the ring can be further substituted.

In a further aspect, the present invention provides pharmaceutical compositions comprising bicyclic aryl nitrone compounds and a pharmaceutical carrier, excipient or diluent. In this aspect of the invention, the pharmaceutical composition can comprise a bicyclic aryl nitrone compound described above.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease or autoimmune disorders, which method comprises administering an effective amount of one or more of the pharmaceutical compositions just described.

In yet another method of treatment aspect, this invention provides a method of treating a mammal susceptible to or afflicted with a condition that gives rise to pain responses or that relates to imbalances in the maintenance of basal activity of sensory nerves. Nitrone compounds have use as analgesics for the treatment of pain of various geneses or etiology, for example, acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain (such as that associated with gastroesophageal reflex disease, irritable bowel syndrome, inflammatory bowel disease, pancreatitis, and various gynecological and urological disorders); dental pain; and headache (such as migraine, cluster headache and tension headache).

In additional method of treatment aspects, this invention provides methods of treating a mammal susceptible to or afflicted with neurodegenerative diseases and disorders such as, for example, Parkinson's disease, Alzheimer's disease and multiple sclerosis; diseases and disorders which are mediated by or result in neuroinflammation such as, for example, traumatic brain injury, stroke and encephalitis; centrally-mediated neuropsychiatric diseases and disorders such as, for example, depression, mania, bipolar disease, anxiety and schizophrenia; eating disorders, sleep disorders and cognition disorders; epilepsy and seizure disorders; prostate, bladder and bowel dysfunction such as, for example, urinary incontinence, urinary hesitancy, rectal hypersensitivity, fecal incontinence, benign prostatic hypertrophy and inflammatory bowel disease; respiratory and airway diseases and disorders such as, for example, allergic rhinitis, asthma, reactive airway diseases and chronic obstructive pulmonary disease; diseases and disorders which are mediated by or result in inflammation such as, for example, rheumatoid arthritis, osteoarthritis, myocardial infarction, various autoimmune diseases and disorders, uveitis and atherosclerosis; itch/pruritus such as, for example, psoriasis; alopecia (hair loss); obesity; lipid disorders; cancer; high blood pressure; spinal cord injury; and renal disorders. The method comprises administering an effective condition-treating or condition-preventing amount of one or more of the pharmaceutical compositions just described.

In additional aspects, this invention provides methods for synthesizing the aryl, heteroaromatic and bicyclic aryl nitrone compounds of the invention.

### BRIEF DESCRIPTION OF THE DRAWINGS

FIG. 1 is an illustration of representative oxidative synthetic pathways to nitrone compounds of the invention.

### DETAILED DESCRIPTION OF THE INVENTION

Definitions

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope. By way of non-limiting example, such substituents may include e.g. halo (such as fluoro, chloro, bromo), —CN, —CF₃, —OH, —OCF₃, C₂₋₆ alkenyl, C₃₋₆ alkynyl, C₁₋₆ alkoxy, aryl and di-C₁₋₆ alkylamino.

“Acyl” refers to a radical —C(O)R, where R is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a radical —NR′C(O)R, where R′ is hydrogen, alkyl, cycloalkyl, cycloheteroalkyl, aryl, arylalkyl, heteroalkyl, heteroaryl, heteroarylalkyl and R is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)R where R is hydrogen, alkyl, aryl or cycloalkyl.

“Substituted alkenyl” includes those groups recited in the definition of “substituted” herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonylamino” refers to the group —NRC(O)OR′ where R is hydrogen, alkyl, aryl or cycloalkyl, and R′ is alkyl or cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkenylene, alkynyl and alkynylene. Aliphatic groups typically have from 1 or 2 to about 12 carbon atoms.

“Alkyl” refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyls” as defined below.

“Substituted alkyl” includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

“Alkylene” refers to divalent saturated aliphatic hydrocarbyl groups particularly having up to about 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” includes those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkenyl” refers to monovalent olefinically unsaturated hydrocarbyl groups preferably having up to about 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbyl groups particularly having up to about 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” includes those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkanoyl” or “acyl” as used herein refers to the group R—C(O)—, where R is hydrogen or alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Substituted Aryl” includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Fused Aryl” refers to an aryl having two of its ring carbon in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group alkyl-NR′R″, wherein each of R′ and R″ are independently selected from hydrogen and alkyl.

“Arylamino” refers to the group aryl-NR′R″, wherein each of R′ and R″ are independently selected from hydrogen, aryl and heteroaryl.

“Alkoxyamino” refers to a radical —N(H)OR where R represents an alkyl or cycloalkyl group as defined herein.

“Alkoxycarbonyl” refers to a radical —C(O)-alkoxy where alkoxy is as defined herein.

“Alkylarylamino” refers to a radical —NRR′ where R represents an alkyl or cycloalkyl group and R′ is an aryl as defined herein.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR where R is an alkyl or cycloalkyl group as defined herein that may be optionally substituted as defined herein. Representative examples include, but are not limited to, methylthio, ethylthio, propylthio, butylthio, and the like.

“Amino” refers to the radical —NH₂.

“Substituted amino” includes those groups recited in the definition of “substituted” herein, and particularly refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Aminocarbonyl” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalky, or where the R groups are joined to form an alkylene group.

“Arylalkyloxy” refers to an —O-arylalkyl radical where arylalkyl is as defined herein.

“Arylamino” means a radical —NHR where R represents an aryl group as defined herein.

“Aryloxycarbonyl” refers to a radical —C(O)—O-aryl where aryl is as defined herein.

“Arylsulfonyl” refers to a radical —S(O)₂R where R is an aryl or heteroaryl group as defined herein.

“Azido” refers to the radical —N₃.

“Carbamoyl” refers to the radical —C(O)N(R)₂ where each R group is independently hydrogen, alkyl, cycloalkyl or aryl, as defined herein, which may be optionally substituted as defined herein.

“Carboxy” refers to the radical —C(O)OH.

“Carboxyamino” refers to the radical —N(H)C(O)OH.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Substituted cycloalkyl” includes those groups recited in the definition of “substituted” herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Substituted cycloalkenyl” includes those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Fused Cycloalkenyl” refers to a cycloalkenyl having two of its ring carbon atoms in common with a second aliphatic or aromatic ring and having its olefinic unsaturation located to impart aromaticity to the cycloalkenyl ring.

“Cyanato” refers to the radical —OCN.

“Cyano” refers to the radical —CN.

“Dialkylamino” means a radical —NRR′ where R and R′ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Ethenyl” refers to substituted or unsubstituted —(C═C)—.

“Ethylene” refers to substituted or unsubstituted —(C—C)—.

“Ethynyl” refers to —(C≡C)—.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to, —X, —R¹⁴, —O—, ═O, —OR¹⁴, —SR¹⁴, —S⁻, ═S, —NR¹⁴R¹⁵, ═NR¹⁴, —CX₃, —CF₃, —CN, —OCN, —SCN, —NO, —NO₂, ═N₂, —N₃, —S(O)₂O⁻, —S(O)₂OH, —S(O)₂R¹⁴, —OS(O₂)O⁻, —OS(O)₂R¹⁴, —P(O)(O—)₂, —P(O)(OR¹⁴)(O⁻), —OP(O)(OR¹⁴)(OR¹⁵), —C(O)R¹⁴, —C(S)R⁴, —C(O)OR⁴, —C(O)NR⁴R¹⁵, —C(O)O⁻, —C(S)OR¹⁴, —NR¹⁶C(O)NR¹⁴R¹⁵, —NR¹⁶C(S)NR¹⁴R⁵, —NR¹⁷C(NR¹⁶)NR¹⁴R⁵ and —C(NR¹⁶)NR¹⁴R¹⁵, where each X is independently a halogen; each R¹⁴, R¹⁵, R¹⁶ and R¹⁷ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR¹⁸R¹⁹, —C(O)R¹⁸ or —S(O)₂R¹⁸ or optionally R¹⁸ and R¹⁹ together with the atom to which they are both attached form a cycloheteroalkyl or substituted cycloheteroalkyl ring; and R¹⁸ and R¹⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

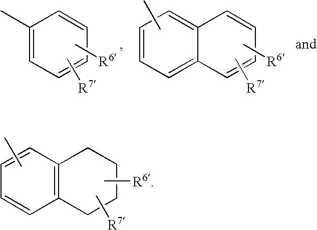

In these formulae one of R^{6′} and R^{7′} may be hydrogen and at least one of R^{6′} and R^{7′} is each independently selected from alkyl, alkenyl, alkynyl, cycloheteroalkyl, alkanoyl, alkoxy, aryloxy, heteroaryloxy, alkylamino, arylamino, heteroarylamino, NR¹⁰COR¹¹, NR¹⁰SOR¹¹, NR¹⁰SO₂R¹⁴, COOalkyl, COOaryl, CONR¹⁰R¹¹, CONR¹⁰OR¹¹, NR¹⁰R¹¹, SO₂NR¹⁰R¹¹, S-alkyl, S-alkyl, SOalkyl, SO₂alkyl, Saryl, SOaryl, SO₂aryl; or R^{6′} and R^{7′} may be joined to form a cyclic ring (saturated or unsaturated) from 5 to 8 atoms, optionally containing one or more heteroatoms selected from the group N, O or S. R¹⁰, R¹¹, and R¹² are independently hydrogen, alkyl, alkenyl, alkynyl, perfluoroalkyl, cycloalkyl, cycloheteroalkyl, aryl, substituted aryl, heteroaryl, substituted or hetero alkyl or the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Particlar heteroaryl groups are those derived from thiophen, pyrrole, benzothiophene, benzofuran, indole, pyridine, quinoline, imidazole, oxazole and pyrazine.

Examples of representative heteroaryls include the following: 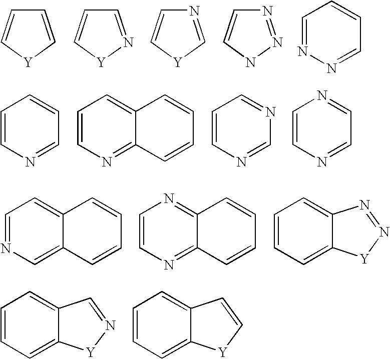
wherein each Y is selected from carbonyl, N, NR⁴, O, and S.

Examples of representative cycloheteroalkyls include the following 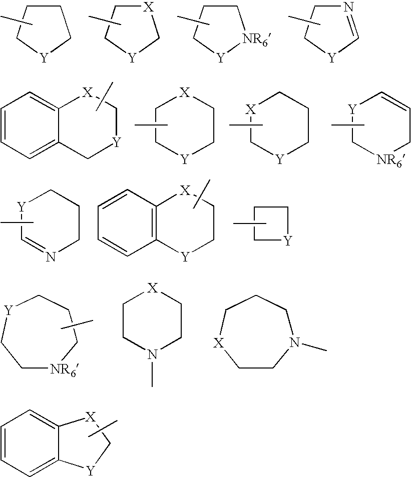
wherein each X is selected from CR⁴₂, NR⁴, O and S; and each Y is selected from NR⁴, O and S, and where R^{6′} is R².

Examples of representative cycloheteroalkenyls include the following: 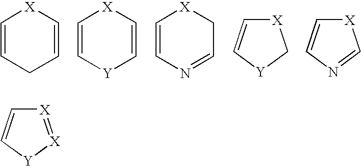
wherein each X is selected from CR⁴, NR⁴, O and S; and each Y is selected from carbonyl, N, NR⁴, O and S.

Examples of representative aryl having hetero atoms containing substitution include the following: 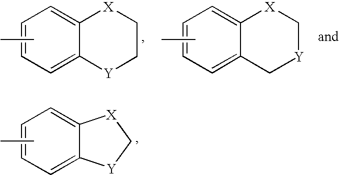
wherein each X is selected from C—R⁴, CR⁴₂, NR⁴, O and S; and each Y is selected from carbonyl, NR⁴, O and S.

“Hetero substituent” refers to a halo, O, S or N atom-containing functionality that may be present as an R⁴ in a R⁴C group present as substituents directly on A, B, W, X, Y or Z of the compounds of this invention or may be present as a substituent in the “substituted” aryl and aliphatic groups present in the compounds.

Examples of hetero substituents include:
-halo,
—NO₂, —NH₂, —NHR, —N(R)₂,
—NRCOR, —NRSOR, —NRSO₂R, OH, CN, CO₂R,
—CO₂H,
—R—OH, —O—R, —COOR,
—CON(R)₂, —CONROR,
—SO₂H, —R—S, —SO₂N(R)₂,
—S(O)R, —S(O)₂R, wherein each R is independently an aryl or aliphatic, optionally with substitution. Among hetero substituents containing R groups, preference is given to those materials having aryl and alkyl R groups as defined herein. Preferred hetero substituents are those listed above.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples: 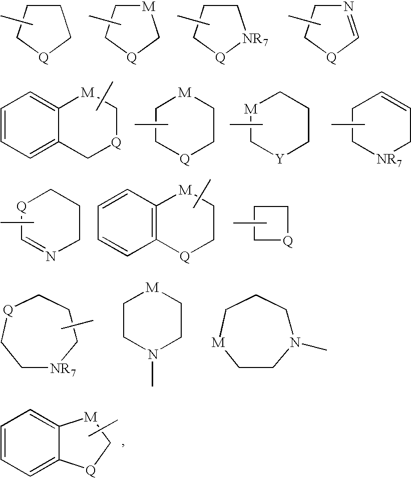
optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR², O, or S; Q is O, NR² or S. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Dihydroxyphosphoryl” refers to the radical —PO(OH)₂.

“Substituted dihydroxyphosphoryl” includes those groups recited in the definition of “substituted” herein, and particularly refers to a dihydroxyphosphoryl radical wherein one or both of the hydroxyl groups are substituted. Suitable substituents are described in detail below.

“Aminohydroxyphosphoryl” refers to the radical —PO(OH)NH₂.

“Substituted aminohydroxyphosphoryl” includes those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

“Thioalkoxy” refers to the group —SR where R is alkyl.

“Substituted thioalkoxy” includes those groups recited in the definition of “substituted” herein, and particularly refers to a thioalkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as R—(O₂)S— wherein R is any substituent described herein. “Aminosulfonyl” or “Sulfonamide” refers to the radical H₂N(O₂)S—, and “substituted aminosulfonyl” “substituted sulfonamide” refers to a radical such as R₂N(O₂)S— wherein each R is independently any substituent described herein.

“Sulfone” refers to the group —SO₂R. In particular embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

“Subject” includes humans. The terms “human,” “patient” and “subject” are used interchangeably herein.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the C₁ to C₈ alkyl, C₂-C₈ alkenyl, aryl, C₇-C₁₂ substituted aryl, and C₇-C₁₂ arylalkyl esters of the compounds of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)— or (S)— stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Aryl, Heteroaromatic and Bicyclic Aryl Nitrone Compounds

The present invention provides aryl, heteroaromatic and bicyclic aryl nitrone compounds useful for preventing and/or treating arthritis, Parkinson's disease, Alzheimer's disease, stroke, uveitis, asthma, myocardial infarction, pain syndromes (acute and chronic or neuropathic), traumatic brain injury, acute spinal cord injury, neurodegenerative disorders, alopecia (hair loss), inflammatory bowel disease and autoimmune disorders or conditions in mammals.

In certain embodiments, the present invention provides aryl, heteroaromatic and bicyclic aryl nitrone compounds according to formula (I): 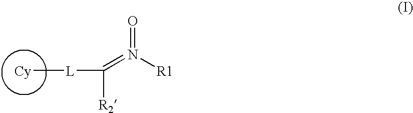
or a pharmaceutically acceptable salt, prodrugs or solvate thereof, wherein:
L is —[C(R²)₂]ₘ—X′—[C(R³)₂]ₙ—; m is an integer from 0 to 6; n is an integer from 1 to 6;
X′ is selected from no atom, NR2, O, S, SO and SO₂; Cy is substituted or unsubstituted (C₁-C₆)cycloalkyl, substituted or unsubstituted aryl, substituted or unsubstituted heteroaryl, substituted or unsubstituted cycloheteroalkyl, bicycloalkenyl, bicycloheteroalkenyl, bicycloaryl, or bicycloheteroaryl ring; provided that when X′ is no atom then Cy is substituted or unsubstituted heteroaryl;
R¹ is selected from substituted or unsubstituted aliphatic, substituted or unsubstituted alkyl, substituted or unsubstituted heteroalkyl, substituted or unsubstituted aryl, substituted or unsubstituted heteroaryl, substituted or unsubstituted aralkyl, substituted or unsubstituted heteroaralkyl;
R² is hydrogen, substituted or unsubstituted (C₁-C₆)alkyl, substituted or unsubstituted (C₁-C₆)cycloalkyl, substituted or unsubstituted aryl, substituted or unsubstituted heteroaryl, and substituted or unsubstituted aralkyl;
R^{2′} is hydrogen, substituted or unsubstituted (C₁-C₆)alkyl, substituted or unsubstituted (C₁-C₆)cycloalkyl, substituted or unsubstituted aryl, substituted or unsubstituted heteroaryl, and substituted or unsubstituted aralkyl;
each R³ is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl, and any two R³s may join together to form a cycloalkyl, cycloheteroalkyl ring; and
one of R²s and one of R³s on carbon atoms adjacent to X′ may join together to form a heterocyclic ring of 5-7 atoms.

In certain embodiments, the present invention provides aryl, heteroaromatic and bicyclic aryl nitrone compounds according to formula (I) and wherein Cy is 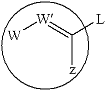
and wherein:
for aryl nitrones, W and Z are joined to form a substituted or unsubstituted cycloalkenyl or aryl ring of 5 to 8 atoms;
for heteroaromatic nitrones, W and Z are joined to form a substituted or unsubstituted cycloheteroalkenyl or heteroaryl ring of 5 to 8 atoms; and
for bicyclic aryl nitrones, W and Z are joined to form a bicycloalkenyl, bicycloheteroalkenyl, bicycloaryl, or bicycloheteroaryl ring of 8 to 11 atoms.

In preferred embodiments, the nitrone compounds according to formula (I) do not encompass any of Compounds 1-26. In particular embodiments, the nitrone compounds according to formula (I) do not encompass any salt of Compounds 1-26. In further embodiments, the nitrone compounds according to formula (I) do not encompass any isomer, diastereomer or enantiomer of Compounds 1-26. Compounds 1-26 follow:
1. Benzenemethanamine, N-[(1S)-2-methyl-1-[(Z)-[oxido(phenylmethyl)imino]methyl]propyl]-N-(phenylmethyl)-
2. Benzeneethanamine, .alpha.-[(Z)-[oxido(phenylmethyl)imino]methyl]-N,N-bis(phenylmethyl)-, (.alpha.S)-
3. Benzenemethanamine, N-[(2S)-2,3-bis(phenylmethoxy)propylidene]-, N-oxide, [N(Z)]-
4. Benzenemethanamine, N-[(2R)-3-fluoro-2-(phenylmethoxy)propylidene]-, N-oxide, [N(Z)]-
5. Benzenemethanamine, N-[(1S)-1-methyl-2-[oxido(phenylmethyl)imino]ethyl]-N-(phenylmethyl)-
6. Glycine, N-[3-(phenylmethoxy)propylidene]-, 1,1-dimethylethyl ester, N-oxide
7. Benzenemethanamine, N-[2-(phenylmethoxy)ethylidene]-, N-oxide,
8. Benzenemethanamine, N-[1-methyl-2-[oxido(phenylmethyl)imino]ethyl]-N-(phenylmethyl)-, [S-(Z)]-
9. Carbamic acid, [2-[oxido(phenylmethyl)imino]-1-(phenylmethyl)ethyl](phenylmethyl)-, 1,1-dimethylethyl ester, [S-(Z)]-
10. Carbamic acid, [1-methyl-2-[oxido(phenylmethyl)imino]ethyl](phenylmethyl)-, 1,1-dimethylethyl ester, [S-(Z)]-
11. Benzenemethanamine, N-[2-(phenylmethoxy)ethylidene]-, N-oxide
12. Benzenemethanamine, N-[2-(phenylmethoxy)propylidene]-, N-oxide, [S-(Z)]-
13. Benzenemethanamine, N-[3-(phenylmethoxy)propylidene]-, N-oxide, (Z)-
14. 2-Butanone, 4-[[1-methyl-2-(methyloxidoimino)ethyl](phenylmethyl)amino]-, (S)-
15. 2-Butanone, 4-[[1-methyl-2-[(phenylmethyl)imino]ethyl](phenylmethyl) amino]-, N-oxide,
16. 2-Butanone, 4-[[2-[(1,1-dimethylethyl)oxidoimino]-1-methylethyl](phenylmethyl)amino]-, (S)-
17. Benzenemethanamine, N-[2-[(1-phenyl-3-butenyl)oxy]ethylidene]-, N-oxide
18. Benzenemethanamine, N-[3-[(4-methoxyphenyl)methoxy]propylidene]-, N-oxide, (Z)-
19. Acetamide, 2-[[4-(dimethylamino)phenyl]imino]-N-2-naphthalenyl-N-phenyl-, N-oxide
20. Acetamide, 2-[[4-(dimethylamino)phenyl]oxidoimino]-N-1-naphthalenyl-N-phenyl-
21. Acetamide, N-1-naphthalenyl-2-(oxidophenylimino)-N-phenyl-
22. 2-Propanol, 1-[(1-methylethyl)imino]-3-(1-naphthalenyloxy)-, N-oxide,
23. Acetamide, 2-[[4-(dimethylamino)phenyl]oxidoimino]-N-(1-methoxy-2-naphthalenyl)-
24. Acetamide, N-1-naphthalenyl-2-(oxidophenylimino)-
25. Methanamine, N-[2-[(4,6-dimethoxy-2-pyrimidinyl)oxy]-3-methylbutylidene]-, N-oxide
26. Methanamine, N-[2-[(4,6-dimethoxy-2-pyrimidinyl)oxy]-3,3-dimethylbutylidene]-, N-oxide

In certain embodiments, the present invention provides aryl nitrone compounds according to formula (I) and wherein Cy is 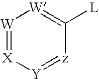
wherein:
W, W′, X, Y and Z are each independently C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio.

In certain embodiments, the present invention provides heteroaromatic nitrone compounds according to formula (I) and wherein Cy is 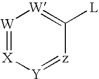
wherein:
m′ of W, W′, X, Y and Z is N and the remainder are each independently C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and m′ is an integer from 0 to 3.

In certain embodiments, the present invention provides heteroaromatic nitrone compounds according to formula (I) and wherein Cy is 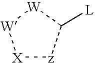
wherein:
W, W′, X, and Z is independently selected from C—R⁴, O, S, SO, SO₂, NR2′ and N;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted bond is single or double bond.

In certain embodiments, the present invention provides bicyclic aryl nitrone compounds according to formula (I) and wherein Cy is 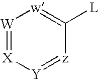
wherein W, W′, X, Y and Z are members of a cycloalkenyl, aryl, cycloheteroalkenyl or heteroaryl ring; and
any adjacent pair of W, W′, X, Y and Z are further joined to form, together with the cycloalkenyl, aryl, cycloheteroalkenyl or heteroaryl ring comprising W, W′, X, Y and Z, the bicycloalkenyl, bicycloheteroalkenyl, bicycloaryl, or bicycloheteroaryl ring.

In certain embodiments, the present invention provides bicyclic aryl nitrone compounds according to formula (I) and wherein Cy is selected from substituted or unsubstituted: 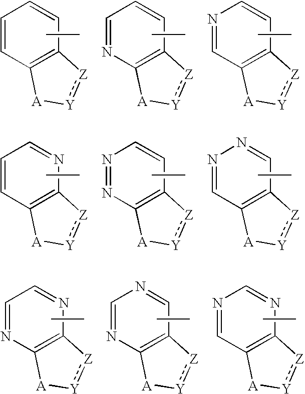
and wherein A, Y and Z are independently selected from C═O, CR4, NR2, O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and the dotted line represents single or double bond.

In certain embodiments, the present invention provides bicyclic aryl nitrone compounds according to formula (I) and wherein Cy is selected from substituted or unsubstituted: 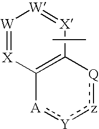
wherein W, W′, X and X′ are each independently NR2 or C—R4;
Y and Z are each independently C—R4 or carbonyl;
A and Q are independently selected from C—R4, NR2, O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and the dotted line represents single or double bond.

In certain embodiments, the present invention provides bicyclic aryl nitrone compounds according to formula (I) and wherein Cy is selected from substituted or unsubstituted:

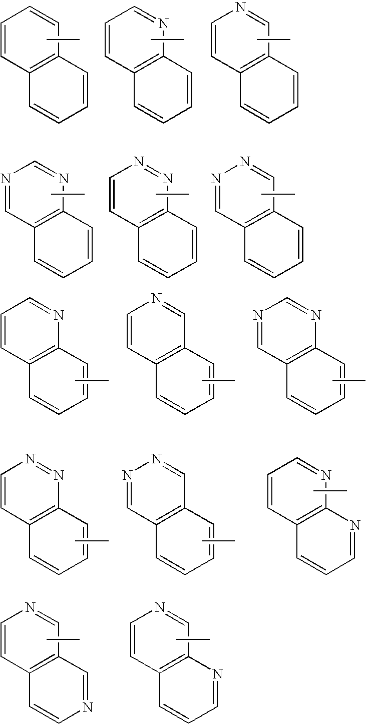

In certain embodiments, the present invention provides bicyclic aryl nitrone compounds according to formula (I) and wherein Cy is selected from substituted or unsubstituted: 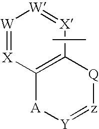
wherein W, W′, X and X′ are each independently NR2 or C—R4;
Y and Z are each independently C—R4;
A and Q are independently selected from C—R4, NR2, O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond.

In certain embodiments, the present invention provides bicyclic aryl nitrone compounds according to formula (I) and wherein Cy is selected from substituted or unsubstituted: 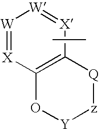
wherein W, W′, X and X′ are each independently NR2 or C—R4;
Y and Z are each independently C—R4 or carbonyl;
Q is selected from NR2, O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond.

In certain embodiments, the present invention provides bicyclic aryl nitrone compounds according to formula (I) and wherein Cy is selected from substituted or unsubstituted: 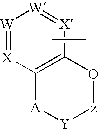
wherein W, W′, X and X′ are each independently NR2 or C—R4;
Y and Z are each independently C—R4 or carbonyl;
A is selected from NR2, O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond.

In certain embodiments, the present invention provides bicyclic aryl nitrone compounds according to formula (I) and wherein Cy is selected from substituted or unsubstituted: 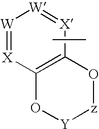
wherein W, W′, X and X′ are each independently NR2 or C—R4;
Y and Z are each independently C—R4 or carbonyl;
   each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and the dotted line represents single or double bond.

In certain embodiments, the present invention provides nitrone compounds according to formula (I) and wherein R^{2′} is hydrogen.

In certain embodiments, the present invention provides nitrone compounds according to formula (I) and wherein L is —[C(R²)₂]ₘ—X′—[C(R³)₂]ₙ—.

In further embodiments, the present invention provides nitrone compounds according to formula (I) and wherein L is —[CH₂]ₘ—X′—[CH₂]ₙ—.

In further embodiments, the present invention provides nitrone compounds according to formula (I) and wherein L is selected from —CH2—, —(CH2)2—, —(CH2)3—, —(CH2)4—, —(CH2)5—, —OCH2—, —O(CH2)2—, —O(CH2)3—, —O(CH2)4—, —O(CH2)5—, —SCH2—, S(CH2)2—, —S(CH2)3—, —S(CH2)4—, —S(CH2)5—, —SOCH2—, —SO(CH2)2—, —SO(CH2)3—, —SO(CH2)4—, —SO(CH2)5—, —N(Me)CH2—, —SO2CH2—, —SO2(CH2)2—, —SO2(CH2)3—, —SO2(CH2)4—, —SO2(CH2)5—, —N(Me)(CH2)2—, —N(Me)(CH2)3—, —N(Me)(CH2)4—, —N(Me)(CH2)5—, —CH2—O—CH2—, —CH2—O—(CH2)2—, —CH2—O—(CH2)3—, —(CH2)2—O—CH2—, —(CH2)2—O—(CH2)2—, —(CH2)3—O—CH2—, —(CH2)3—O—(CH2)2—, —CH2—S—CH2—, —CH2—S—(CH2)2—, —CH2—S—(CH2)3—, —(CH2)2—S—CH2—, —(CH2)2—S—(CH2)2—, —(CH2)3—S—CH2—, —(CH2)3—S—(CH2)2—, —CH2—SO—CH2—, —CH2—SO—(CH2)2—, —CH2—SO—(CH2)3—, —(CH2)2—SO—CH2—, —(CH2)2—SO—(CH2)2—, —(CH2)3—SO—CH2—, —(CH2)3—SO—(CH2)2—, —CH2—SO2—CH2—, —CH2—SO2—(CH2)2—, —CH2—SO2—(CH2)3—, —(CH2)2—SO2-CH2—, —(CH2)2—SO2—(CH2)2—, —(CH2)3—SO2—CH2—, —(CH2)3—SO2—(CH2)2—, —CH2—N(Me)-CH2—, —CH2—N(Me)-(CH2)2—, —CH2—N(Me)-(CH2)3—, —(CH2)2—N(Me)-CH2—, —(CH2)2—N(Me)-(CH2)2—, —(CH2)3—N(Me)—CH2—, and —(CH2)3—N(Me)-(CH2)2—.

In certain embodiments, the present invention provides nitrone compounds according to formula (I) and wherein R¹ is t-butyl.

In certain embodiments, the present invention provides nitrone compounds according to formula (I) and wherein R¹ is cyclohexyl.

In certain embodiments, the present invention provides nitrone compounds according to formula (I) and wherein R¹ is benzyl.

Among the aryl nitrones described above by formula (I), there is a general preference for compounds wherein W and Z are joined to form a 6-membered aryl ring.

Among the heteroaromatic nitrones described above by formula (I), there is a general preference for compounds wherein W and Z are joined to form a 6-membered heteroaryl ring. However, the heteroaryl ring can be any 5- to 8-membered heteroaryl ring known to those of skill in the art, including the exemplary heteroaryl rings described in the Definitions section (Section 5.1) above. In certain embodiments, the heteroaryl ring is a pyridine, pyrimidine, furan, thiophene or pyrrole ring.

Referring to bicyclic aryl nitrones of formula (I), in certain embodiments R¹ is substituted with a group other than phenyl, substituted phenyl or methyl. In other embodiments R¹ is substituted with a group other than phenyl, substituted phenyl or lower alkyl. For instance, R¹ can be substituted or unsubstituted heteroalkyl, substituted or unsubstituted alkyl, substituted or unsubstituted heteroaryl, substituted or unsubstituted aralkyl, or substituted or unsubstituted heteroaralkyl.

Also referring to bicyclic aryl nitrones of formula (I), in certain embodiments R^{2′} can be substituted with a group other than hydrogen. For instance, R^{2′} can be substituted or unsubstituted (C₁-C₆)alkyl, substituted or unsubstituted (C₁-C₆)cycloalkyl, substituted or unsubstituted aryl, or substituted or unsubstituted aralkyl.

Referring again to bicyclic aryl nitrones of formula (I), in certain embodiments W and Z are joined to form a six-membered ring that is fused to a second ring. The second ring can be, for instance, a five- or six-membered ring and can contain heteroatom(s). The second ring can be fused to any adjacent pair of atoms in the first ring.

Also referring to bicyclic aryl nitrones of formula (I), in certain embodiments W and Z are joined to form a seven-membered ring that is fused to a second ring. The second ring can be, for instance, a five-membered ring and can contain heteroatom(s). The second ring can be fused to any adjacent pair of atoms in the first ring. For example, the bicyclic aromatic ring can be azulene.

In certain embodiments of aryl and heteroaromatic nitrones of formula (I), W and X of Cy is C—R⁵ and R5 is independently selected from hydrogen, —SR9, SO₂R9-SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹. While the R⁵ substituents at W and X can vary independently, in certain embodiments both R⁵ s are identical. In particular embodiments, R⁵ are identical when it is SO2R9 or SO3H.

Among the nitrone compounds of formula (I) there is a preference for R^{2′} to be hydrogen, alkyl, heteroalkyl, aralkyl or aryl, with or without further substitution. Hydrogen is a most preferred R^{2′} group.

There is a preference for the one or more R⁴ groups to be hydrogen.

There is a preference for R⁵ to be hydrogen, —SR⁹, —SO2R9, —SO₂NR⁷R⁸, SO₃R⁹, —CONR⁷R⁸, —NR⁷R⁸, —OH or —CO₂R⁹. More preferred R⁵ groups are hydrogen, —SO2R9-SO₂NR⁷R⁸, —SO₃R⁹, —CONR⁷R⁸ and —CO₂R⁹.

In the heteroaromatic nitrone compounds of the invention, the atom designated by X can be substituted or unsubstituted, especially in compounds where X is a carbon or a heteroatom with a free valence. In certain embodiments, X can be substituted with any group other than hydrogen. For instance, X can be substituted with —SR⁹, —SO2R9, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹.

Referring to heteroaromatic nitrone compounds of formula (I), in some embodiments for Cy the six-membered heteroaryl ring contains one nitrogen atom, and in other embodiments the heteroaryl ring contains two nitrogen atoms. In further embodiments the ring contains three nitrogen atoms.

When the heteroaryl ring (Cy) of formula (I) contains two nitrogen atoms, the two nitrogen atoms can be at any of W, X, Y and Z. For instance, the two nitrogen atoms can be at W and X, at W and Y, at W and Z, at X and Y, at X and Z, or at Y and Z.

Among the bicyclic aryl nitrone compounds described by formula (I), there is a general preference for compounds wherein W and Z are joined to form a 6-membered aryl or heteroaryl ring fused to a 5- or 6-membered cycloalkyl, cycloheteroalkyl, aryl or heteroaryl ring.

Also among the bicyclic aryl nitrone compounds of the formulas above, there is a general preference for R¹ to be alkyl, cycloalkyl, aryl or aralkyl, preferably alkyl and particularly lower alkyl. Lower alkyls having branching at the 1-position carbon, for example, cyclopropyl, isopropyl, sec-butyl, tert-butyl, cyclobutyl, 1-methylcycloprop-1-yl, sec-pentyl, tert-pentyl, cyclopentyl, 1-methylcyclobut-1-yl and the like are preferred over non-branched equivalents. tert-Butyl is a most preferred R¹ group.

There is a preference for R^{2′} to be hydrogen, alkyl, heteroalkyl, aralkyl or aryl, with or without further substitution. Hydrogen is a most preferred R^{2′} group.

There is a preference for the one or more R⁴ groups to be hydrogen.

There is a preference for R⁵ to be hydrogen, —SR⁹, —SO₂NR⁷R⁸, —SO₃R⁹, —SO2R9, —CONR⁷R⁸, —NR⁷R⁸, —OH, —PO(OR⁹)₂ or —CO₂R⁹. More preferred R⁵ groups are hydrogen, —SO₂NR⁷R⁸, —SO₃R⁹, —CONR⁷R⁸ and —CO₂R⁹.

In the bicyclic aryl nitrone compounds of the invention, the atom designated by X can be substituted or unsubstituted, especially in compounds where X is a carbon or a heteroatom with a free valence. In certain embodiments, X can be substituted with any group other than hydrogen. For instance, X can be substituted with hydrogen, —SR⁹, —SO₂NR⁷R⁸, —SO₃R⁹, —CONR⁷R⁸, —NR⁷R⁸, —OH, —PO(OR⁹)NR⁷R⁸, —PO(OR⁹)₂ or —CO₂R⁹.

Derivatives of Aryl, Heteroaromatic and Bicyclic Aryl Nitrone Compounds

In certain aspects, the present invention provides prodrugs and derivatives of: aryl nitrone compounds of formula (I). Other derivatives of the aryl, heteroaromatic and bicyclic aryl nitrone compounds of this invention have activity in both their acid and acid-derivative forms. An acid-sensitive form often offers advantages of solubility, tissue compatibility or delayed release in the mammalian organism (See H. Bundgard, 1985, Design of Prodrugs, Elsevier, Amsterdam, pp. 7-9, 21-24). Prodrugs include acid derivatives well known to practitioners of the art, such as, for example, esters prepared by reaction of the parent acid with a suitable alcohol, amides prepared by reaction of the parent acid compound with a substituted or unsubstituted amine, acid anhydrides and mixed anhydrides. Simple aliphatic or aromatic esters, amides and anhydrides derived from acidic groups pendant on the compounds of this invention are preferred prodrugs. In some cases it is desirable to prepare double ester-type prodrugs such as (acyloxy)alkyl esters or ((alkoxycarbonyl)oxy)alkyl esters. Preferred are the C₁-C₈ alkyl, C₂-C₈ alkenyl, aryl, C₇-C₁₂ substituted aryl and C₇-C₁₂ arylalkyl esters of the compounds of the invention.

Pharmaceutical Compositions

When employed as pharmaceuticals, the aryl, heteroaromatic and bicyclic aryl nitrone compounds of this invention are typically administered in the form of a pharmaceutical composition. Such compositions can be prepared in a manner well known in the pharmaceutical art and typically comprise a pharmaceutically acceptable carrier and a pharmaceutically effective amount of at least one active compound.

In general, the aryl, heteroaromatic and bicyclic aryl nitrone compounds of this invention are administered in a pharmaceutically effective amount. The amount of the compound actually administered will typically be determined by a physician in light of relevant circumstances, including the condition to be treated, the severity of the patient's symptoms, the chosen route of administration, the actual compound administered, the age, weight, and response of the patient to the treatment, and the like.

The pharmaceutical compositions of this invention can be administered by a variety of routes, including oral, rectal, transdermal, subcutaneous, intravenous, intramuscular and intranasal. Depending on the intended route of delivery, the compounds of this invention are preferably formulated as injectable or oral compositions or, for transdermal administration, as salves, lotions or patches.

The compositions for oral administration can take the form of bulk powders or bulk liquid solutions or suspensions. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules and the like in the case of solid compositions. In such compositions, the active nitrone compound of the invention is usually a minor component (from about 0.1 to about 50% by weight or preferably from about 1 to about 40% by weight) with the remainder being various vehicles or carriers and processing aids helpful for creating the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose; a disintegrating agent such as alginic acid, Primogel or corn starch; a lubricant such as magnesium stearate; a glidant such as colloidal silicon dioxide; a sweetening agent such as sucrose or saccharin; and a flavoring agent such as peppermint, methyl salicylate or orange flavoring.

Injectable compositions are typically based on injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, more preferably from about 0.1 to about 10% by weight, and even more preferably from about 0.5 to about 15% by weight. When formulated as an ointment, the active ingredients will typically be combined with either a paraffinic or a water-miscible ointment base. Alternatively, the active ingredients may be formulated in a cream with, for example, an oil-in-water cream base. Such transdermal formulations are well known in the art and generally include additional ingredients to enhance the dermal penetration or stability of the active ingredients or the formulation. All such known transdermal formulations and ingredients are included within the scope of this invention.

The aryl, heteroaromatic and bicyclic aryl nitrone compounds of this invention can also be administered by a transdermal device. Accordingly, transdermal administration can be accomplished using a patch either of the reservoir or porous membrane type or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference in its entirety.

The aryl, heteroaromatic and bicyclic aryl nitrone compounds of this invention can also be administered in sustained-release forms or from sustained-release drug delivery systems. A description of representative sustained-release materials can be found in Remington's Pharmaceutical Sciences.

The following formulation examples illustrate representative pharmaceutical compositions of this invention. The present invention, however, is not limited to the following pharmaceutical compositions.

### Formulation 1—Tablets

An aryl, heteroaromatic or bicyclic aryl nitrone compound of formula (I) is admixed as a dry powder with a dry gelatin binder in an approximate 1:2 weight ratio. A minor amount of magnesium stearate is added as a lubricant. The mixture is formed into 240-270 mg tablets (80-90 mg of active nitrone compound per tablet) in a tablet press.

### Formulation 2—Tablets

An aryl, heteroaromatic or bicyclic aryl nitrone compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active nitrone compound) in a tablet press.

### Formulation 3—Capsules

An aryl, heteroaromatic or bicyclic aryl nitrone compound of formula (I) is admixed as a dry powder with a starch diluent in an approximate 1:1 weight ratio. The mixture is filled into 250 mg capsules (125 mg of active nitrone compound per capsule).

### Formulation 4—Liquid

An aryl, heteroaromatic or bicyclic aryl nitrone compound of formula (I) (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor and color are diluted with water and added with stirring. Sufficient water is then added to produce a total volume of 5 mL.

### Formulation 5—Injection

An aryl, heteroaromatic or bicyclic aryl nitrone compound of formula (I) is dissolved or suspended in a buffered, sterile, saline, injectable, aqueous medium to a concentration of approximately 5 mg/ml.

### Formulation 6—Topical

Stearyl alcohol (250 g) and a white petrolatum (250 g) are melted at about 75° C. and then a mixture of an aryl, heteroaromatic or bicyclic aryl nitrone compound of formula (I) (50 g), methylparaben (0.25 g), propylparaben (0.15 g), sodium lauryl sulfate (10 g) and propylene glycol (120 g) dissolved in water (about 370 g) is added. The resulting mixture is stirred until it congeals.

Methods of Treatment

The aryl, heteroaromatic and bicyclic aryl nitrone compounds of the present invention are used as therapeutic agents for the treatment of conditions in mammals. Accordingly, the compounds and pharmaceutical compositions of this invention find use as therapeutics for preventing and/or treating pain, neurological and neurodegenerative, autoimmune and inflammatory diseases or conditions in mammals including humans.

In a method of treatment aspect, this invention provides a method of treating a mammal susceptible to or afflicted with a condition associated with arthritis, uveitis, asthma, myocardial infarction, traumatic brain injury, acute spinal cord injury, alopecia (hair loss), inflammatory bowel disease or autoimmune disorders, which method comprises administering an effective amount of one or more of the pharmaceutical compositions described above.

In yet another method of treatment aspect, this invention provides a method of treating a mammal susceptible to or afflicted with a condition that gives rise to pain responses or relates to imbalances in the maintenance of basal activity of sensory nerves. Nitrone compounds have use as analgesics for the treatment of pain of various geneses or etiology, for example, acute inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy and chemotherapy-induced and other iatrogenic neuropathies); visceral pain (such as that associated with gastroesophageal reflex disease, irritable bowel syndrome, inflammatory bowel disease, pancreatitis and various gynecological and urological disorders); dental pain; and headache (such as migraine, cluster headache and tension headache).

In additional method of treatment aspects, this invention provides methods of treating a mammal susceptible to or afflicted with: neurodegenerative diseases and disorders such as, for example, Parkinson's disease, Alzheimer's disease and multiple sclerosis; diseases and disorders which are mediated by or result in neuroinflammation such as, for example, traumatic brain injury, stroke and encephalitis; centrally-mediated neuropsychiatric diseases and disorders such as, for example, depression, mania, bipolar disease, anxiety and schizophrenia; eating disorders, sleep disorders and cognition disorders; epilepsy and seizure disorders; prostate, bladder and bowel dysfunction such as, for example, urinary incontinence, urinary hesitancy, rectal hypersensitivity, fecal incontinence, benign prostatic hypertrophy and inflammatory bowel disease; respiratory and airway diseases and disorders such as, for example, allergic rhinitis, asthma, reactive airway diseases and chronic obstructive pulmonary disease; diseases and disorders which are mediated by or result in inflammation such as, for example, rheumatoid arthritis, osteoarthritis, myocardial infarction, various autoimmune diseases and disorders, uveitis and atherosclerosis; itch/pruritus such as, for example, psoriasis; alopecia (hair loss); obesity; lipid disorders; cancer; high blood pressure; spinal cord injury; and renal disorders. The methods comprise administering an effective condition-treating or condition-preventing amount of one or more of the pharmaceutical compositions described above.

Injection dose levels range from about 0.1 mg/kg/hour to at least 10 mg/kg/hour, all for from about 1 to about 120 hours and especially from 24 to 96 hours. A preloading bolus of from about 0.1 mg/kg to about 10 mg/kg or more may also be administered to achieve adequate steady-state levels. The maximum total dose is not expected to exceed about 2 g/day for a 40 to 80 kg human patient.

For the prevention and/or treatment of long-term conditions, such as neurodegenerative and autoimmune conditions, the regimen for treatment usually stretches over many months or years, so oral dosing is preferred for patient convenience and tolerance. With oral dosing, one to five and especially two to four and typically three oral doses per day are representative regimens. Using these dosing patterns, each dose provides from about 0.01 to about 20 mg/kg of the active nitrone compound, with preferred doses each providing from about 0.1 to about 10 mg/kg and especially from about 1 to about 5 mg/kg.

Transdermal doses are generally selected to provide similar or lower blood levels than those achieved using injection doses.

When used to prevent the onset of a neurodegenerative, autoimmune or inflammatory condition, the nitrone compounds of this invention would be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The aryl, heteroaromatic and bicyclic aryl nitrone compounds of this invention can be administered as the sole active agent or they can be administered in combination with other agents, including other active nitrone compounds.

General Procedures to Synthesize Aryl, Heteroaromatic and Bicyclic Aryl Nitrone Compounds

The aryl, heteroaromatic and bicyclic aryl nitrones of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by one skilled in the art by routine optimization procedures.

In addition, conventional protecting groups may be necessary to prevent certain functional groups from undergoing undesired reactions, as will be apparent to those skilled in the art. The choice of a suitable protecting group for a particular functional group as well as suitable conditions for protection and deprotection are well known in the art. For example, numerous protecting groups and the conditions for their introduction and removal are described in T. W. Greene and P. G. M. Wuts, 1991, Protecting Groups in Organic Synthesis, Second Edition, Wiley, New York, and references cited therein.

One known method for preparing nitrones is to react a carboxaldehyde derivative with an appropriately substituted hydroxlamine (or an acid addition salt thereof) and to isolate and purify the product by known standard procedures. Such procedures include, but are not limited to, recrystallization, column chromatography and HPLC.

The reaction of an aromatic aldehyde or ketone with a substituted hydroxylamine (or an acid addition salt thereof) in an organic solvent such as methanol, tetrahydrofuran, dichloromethane, benzene or toluene is known to produce the desired aryl, heteroaromatic or bicyclic aryl nitrone (Scheme 1). The reaction may be conducted at ambient temperature or may require heating (e.g., refluxing), and may proceed with or without an organic or inorganic acid as catalyst. Higher temperature may be required when an aromatic ketone is a reactant. The condensation reaction may also be accomplished using microwave-mediated synthesis, which typically employs conditions such as heating to 120° C. for 5-10 min in a sealed tube.

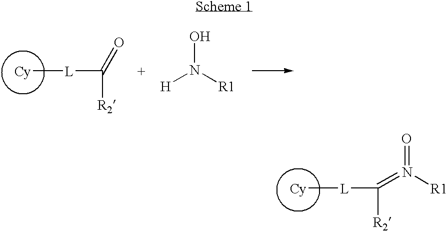

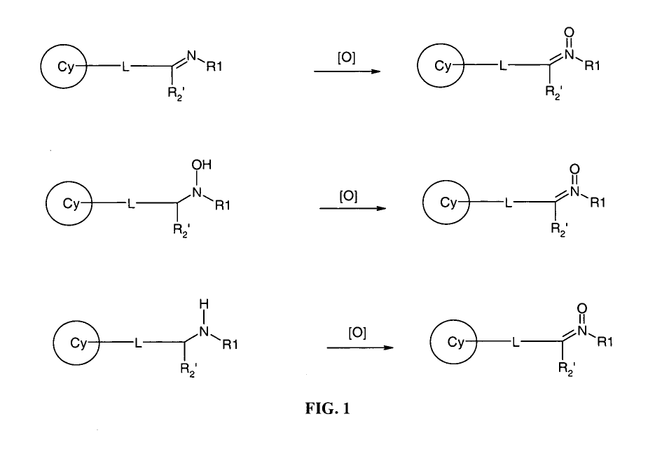

Aryl, heteroaromatic and bicyclic aryl nitrones of the present invention may also be prepared by alternative known methods such as, for example, oxidation of amines, imines or hydroxylamines. FIG. 1 illustrates exemplary oxidative synthetic routes to aryl, heteroaromatic and bicyclic aryl nitrones, respectively.

Representative Syntheses of Starting Aldehydes:

### Intermediate Aldehyde 1

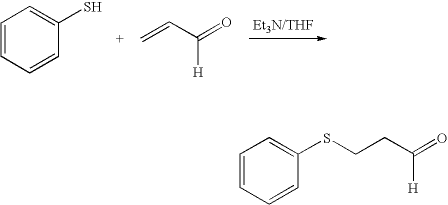

### Synthesis of 3-phenylsulfanyl-propionaldehyde

To a cooled (0° C.) solution of acrolein (1.12 g, 19.98 mmol) in methylene chloride (40 mL) was added triethyl amine (19.98 mmol) followed by thiophenol (2.0 g, 18.15 mmol) slowly dropwise and the mixture stirred at the same temperature for 30 minutes and then warmed to ambient temperature. TLC indicated complete disappearance the starting thiophenol. The mixture was concentrated to dryness, the crude product was chromatographed on silicagel using 1:9, EtOAc and hexane to obtain the title compound as an oil (2.1 g, 70%).

MS: m/z=165 (M−1).

### Intermediate Aldehyde 2

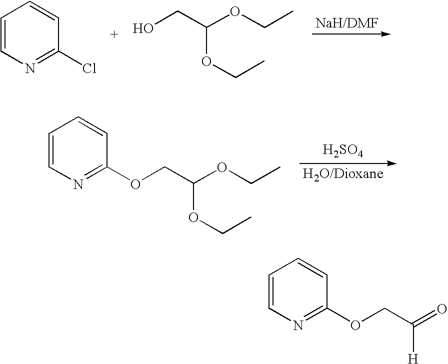

### Synthesis of (pyridin-2-yloxy)-acetaldehyde

To a stirred suspension of sodium hydride (30 mmol) in DMF (6 mL) was added, dropwise, 2-hydroxyacetaldehyde diethylacetal (30 mmol) and the mixture was agitated for 0.5 hrs. A solution of 2-chloropyridine (10 mmol) in DMF (2 mL) was then added and the mixture was stirred at 80° C. for 24 hrs. The reaction was quenched with ice-cold water, extracted with ether (2×150 mL), the combined organic extracts were dried and concentrated to give the crude intermediate acetal which was hydrolyzed as follows.

The crude acetal was dissolved in 10 mL of dioxane and 15 mL of water to which was added Conc. H₂SO₄ (1 mL) and the mixture was heated at 80° C. for 2 hrs. The reaction was then quenched with sat. NaHCO₃, extracted with methylene chloride, the organic extracts were dried and concentrated to give the title compound (0.6 g, 43.6%).

MS: m/z=138 (MH+).

### Intermediate Aldehyde 3

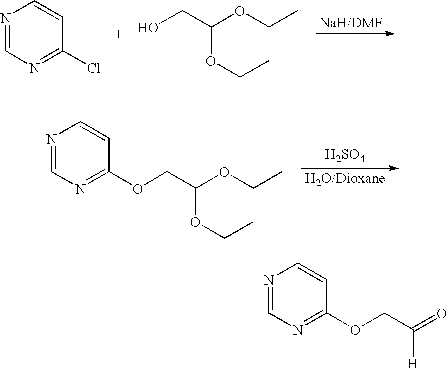

### Synthesis of (pyrimidin-4-yloxy)-acetaldehyde

(pyrimidin-4-yloxy)-acetaldehydes are easily accessible through the exploitation of various substituted halo pyrimidines in a similar reaction sequence described in Intermediate Aldehyde 2.

### Intermediate Aldehyde 4

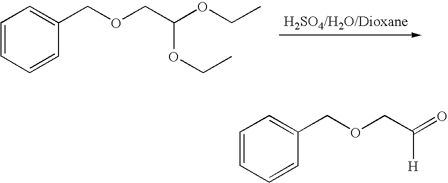

### Synthesis of benzyloxy-acetaldehyde

Commercially available (2,2-Diethoxy-ethoxymethyl)-benzene (2.0 g, 8.92 mM) in a mixture of dioxane (20 ml) and water (10 ml) was treated with 2 ml of conc. H₂SO₄ and the mixture heated at 80° C. for 2 hrs. The reaction was quenched with solid NaHCO₃ and extracted with CH₂Cl₂ (2×100 ml), the combined organic extracts were washed with water, dried and concentrated to obtain the crude product which was chromatographed on silicagel to obtain the title aldehyde as an oil (0.8 g, 58%).

MS: m/z=149 (M−1).

### Intermediate Aldehyde 5

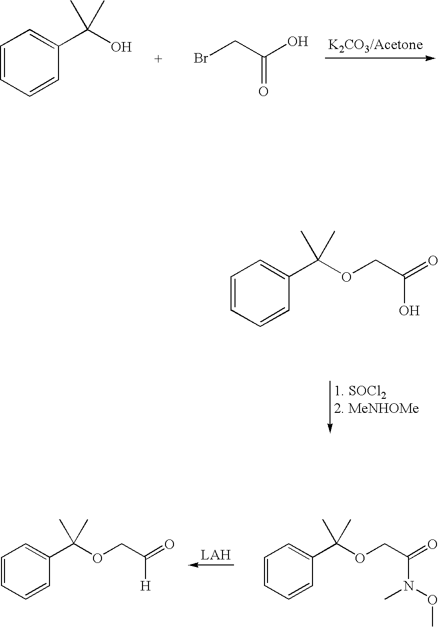

### Synthesis of (1-methyl-1-phenyl-ethoxy)-acetaldehyde

2-Phenyl-propan-2-ol (5.0 g, 36.71 mM) is reacted with α-bromoacetic acid (4.08 g, 27.37 mM) in presence of K₂CO₃/acetone under refluxing conditions for several hrs and the mixture is concentrated to dryness. The crude residue is dissolved in water (100 mL) and carefully acidified using 1N HCl. The precipitate is filtered, washed with water and vacuum dried to obtain the intermediate (1-methyl-1-phenyl-ethoxy)-acetic acid.

(1-methyl-1-phenyl-ethoxy)-acetic acid (2.0 g, 10.3 mM) is heated to reflux in thionyl chloride (25 ml) for 30 minutes and the mixture is concentrated to dryness. The crude acid chloride is then dissolved in methylene chloride to which is added triethylamine (1.25 g, 12.36 mM) followed by N,O-dimethyl hydroxylamine (0.76 g, 10.36 mM) at ambient temperature and the mixture stirred for an additional 12 hrs. The mixture is then concentrated and flash chromatographed on silicagel to obtain N-methoxy-N-methyl-2-(1-methyl-1-phenyl-ethoxy)-acetamide.

The wienreb amide (1.0 g, 4.21 mM) in anhydrous THF is cooled to −75° C. and treated with a solution of LAH (6.32 mM) in THF and the mixture slowly warmed up to ambient temperature. The reaction mixture was further stirred for an additional 2 hrs before being quenched with 1 ml of methanol followed by ice-cold water. The product is extracted with ether washed with water, dried and concentrated to obtain the crude aldehyde which is purified by flash chromatography on silicagel to yield the title compound.

### Intermediate Aldehyde 6

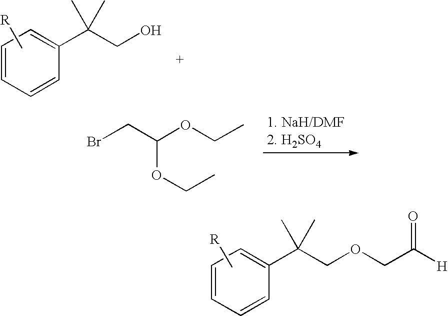

### Synthesis of (2-ethyl-2-substituted-phenyl-propoxy)-acetaldehydes

Commercially available 2-methyl-2-substituted-phenyl-propan-1-ols can be converted to their corresponding (2-methyl-2-substituted-phenyl-propoxy)-acetaldehydes by reacting first with bromoacetaldehyde in presence of NaH followed by the hydrolysis of the intermediate acetals with H₂SO₄.

### Intermediate Aldehyde 7

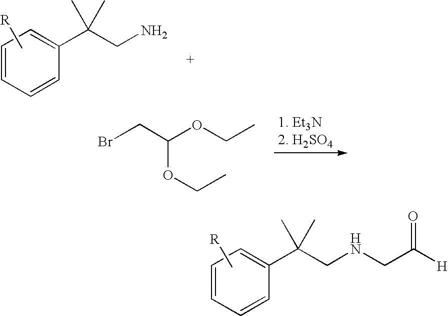

### Synthesis of (2-methyl-2-substituted-phenyl-propylamino)-acetaldehydes

Commercially available 2-methyl-2-substituted-phenyl-propyl amines can be converted to their corresponding (2-methyl-2-substituted-phenyl-propylamino)-acetaldehydes by reacting first with bromoacetaldehyde followed by the hydrolysis of the intermediate acetals with H₂SO₄.

### Intermediate Aldehyde 8

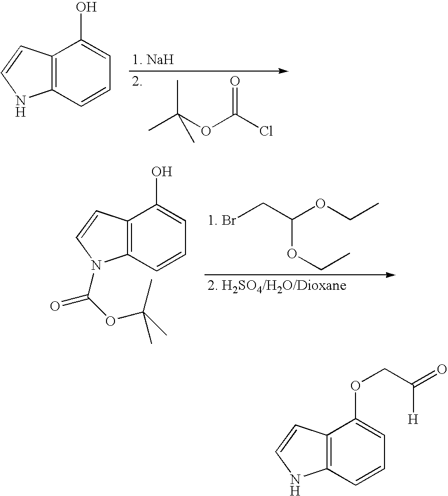

### Synthesis of (1H-indol-4-yloxy)-acetaldehyde

The title compound can conveniently be prepared by selective protection of the nitrogen followed by the reaction of the intermediate with bromoacetaldehyde diethyl acetal in presence of NaH and subsequent sulphuric acid assisted hydrolysis.

### A General Procedure for the Synthesis of Aryl, Heteroaromatic and Bicyclic Aryl Nitrones

A mixture of an appropriate aldehyde or ketone and an appropriate hydroxylamine or acid addition salt thereof (1.5 equiv.) in methanol is stirred at ambient temperature or at elevated (e.g., refluxing) temperature for 6-24 h. Higher temperature may be required when a ketone is a reactant. The progress of the reaction is monitored by chromatographic or spectroscopic techniques such as TLC, LC/MS or ¹H NMR. In some cases excess hydroxylamine or acid addition salt thereof is added to drive the reaction to completion. After the reaction is complete, the mixture is concentrated in vacuo and the crude product is dissolved in ethyl acetate, extracted with water and chromatographed on silica gel to afford the product.

In cases where the sodium salt of a sulfonic acid derivative is desired, the following procedure is employed. The methanolic reaction mixture is set at ambient temperature and treated with sodium methoxide in methanol until the pH of the solution is about 9. Removal of methanol in vacuo followed by precipitation with ether provides the desired sulfonic acid sodium salt derivative.

Nitrone Compounds

### EXAMPLE 1

### N-tert-Butyl-C-(phenoxymethyl)nitrone

Typical Procedure for the Synthesis of Nitrones:

Commercially available phenoxy-acetaldehyde (300 mg, 2.2 mmol) and tert-butyl hydroxyl amine hydrochloride (395 mg, 2.64 mmol) in methanol (5 mL) were stirred together at ambient temperature for 24 hrs. The mixture was then concentrated to dryness, the crude mixture was dissolved in ethyl acetate (15 ml), washed with water (2×20 ml), dried (Na₂SO₄) and concentrated and the crude product was chromatographed on silica gel to obtain the title compound (180 mg, 39.5%).

MS: m/z=208 (MH+).

¹H NMR: (CDCl₃) δ 1.5 (s, 9H); 4.98 (d, 4.0 Hz, 2H); 6.84-7.05 (m, 2H); 7.14 (t, 4.0 Hz, 1H); 7.27-7.34 (m, 3H).

Following the procedure described above in Example 1 or with slight modifications thereof, and following procedures familiar to one of ordinary skill in the art, the following nitrones of Examples 2-10 were prepared by condensation of the corresponding aldehydes with various hydroxylamines.

### EXAMPLE 2

### N-Cyclohexyl-C-(phenoxymethyl)nitrone

The title compound has been prepared according to the procedure described in Example 1.

MS: m/z=234 (MH+).

### EXAMPLE 3

### N-Benzyl-C-(phenoxymethyl)nitrone

The title compound has been prepared according to the procedure described in Example 1.

MS: m/z=242 (MH+).

### EXAMPLE 4

### N-tert-Butyl-C-[(4-methoxy-phenoxy)methyl]nitrone

The title compound has been prepared according to the procedure described in Example 1.

MS: m/z=238 (MH+).

### EXAMPLE 5

### N-Cyclohexyl-C-[(4-methoxy-phenoxy)methyl]nitrone

The title compound has been prepared according to the procedure described in Example 1.

MS: m/z=264 (MH+).

### EXAMPLE 6

### N-Benzyl-C-[(4-methoxy-phenoxy)methyl]nitrone

The title compound has been prepared according to the procedure described in Example 1.

MS: m/z=272 (MH+).

### EXAMPLE 7

### N-tert-Butyl-C-[(2-isopropyl-5-methyl-phenoxy)methyl]nitrone

The title compound has been prepared according to the procedure described in Example 1.

MS: m/z=264 (MH+).

### EXAMPLE 8

### N-Cyclohexyl-C-[(2-isopropyl-5-methyl-phenoxy)methyl]nitrone

The title compound has been prepared according to the procedure described in Example 1.

MS: m/z=290 (MH+).

### EXAMPLE 9

### N-Benzyl-C-[(2-isopropyl-5-methyl-phenoxy)methyl]nitrone

The title compound has been prepared according to the procedure described in Example 1.

MS: m/z=298 (MH+).

### EXAMPLE 10

### N-tert-Butyl-C-[(4-methyl-thiophenoxy)methyl]nitrone

The title compound has been prepared according to the procedure described in Example 1.

MS: m/z=238 (MH+).

### EXAMPLE 11

### N-tert-Butyl-C-(pyridin-2-yloxy)methyl nitrone

(Pyridin-2-yloxy)acetaldehyde (250 mg, 1.82 mmol) and tert-butyl hydroxylamine hydrochloride (326 mg, 2.19 mmol) in methanol (5 mL) were stirred together at ambient temperature for 24 hrs. The mixture was then concentrated to dryness, the crude mixture was dissolved in ethyl acetate (15 ml), washed with water (2×20 ml), dried (Na₂SO₄) and concentrated and the crude product was chromatographed on silica gel to obtain the title compound (70 mg, 18.5%).

MS: m/z=209 (MH+).

Following the procedure described above in Example 11 or with slight modifications thereof, and following procedures familiar to one of ordinary skill in the art, the following nitrones of Examples 11-14 were prepared by condensation of the corresponding aldehydes with various hydroxylamines.

### EXAMPLE 12

### N-Cyclohexyl-C-(pyridin-2-yloxy)methyl nitrone

The title compound has been prepared according to the procedure described in Example 11.

MS: m/z=235 (MH+).

### EXAMPLE 13

### N-Benzyl-C-(pyridin-2-yloxy)methyl nitrone

The title compound has been prepared according to the procedure described in Example 11.

MS: m/z=243 (MH+).

### EXAMPLE 14

### N-tert-Butyl-C-(isoquinolin-2-yloxy)methyl nitrone

The title compound has been prepared according to the procedure described in Example 11.

MS: m/z=259 (MH+).

### EXAMPLE 15

### N-Cyclohexyl-C-(benzyloxy)methyl nitrone

Benzyloxy acetaldehyde (500 mg, 3.33 mmol) and tert-butyl hydroxylamine hydrochloride (596 mg, 4.0 mmol) in methanol (10 mL) were stirred together at ambient temperature for 24 hrs. The mixture was then concentrated to dryness, the crude mixture was dissolved in ethyl acetate (20 ml), washed with water (2×40 ml), dried (Na₂SO₄) and concentrated and the crude product was chromatographed on silica gel to obtain the title compound (363 mg, 49%).

MS: m/z=248 (MH+).

### EXAMPLE 16

### N-Benzyl-C-(benzyloxy)methyl nitrone

The title compound has been prepared according to the procedure described in Example 16.

MS: m/z=256 (MH+).

### EXAMPLE 17

### N-tert-Butyl-C-2-(phenoxy)ethyl nitrone

3-Phenoxy-propionaldehyde (500 mg, 3.33 mmol) and tert-butyl hydroxylamine hydrochloride (596 mg, 4.0 mmol) in methanol (10 mL) were stirred together at ambient temperature for 24 hrs. The mixture was then concentrated to dryness, the crude mixture was dissolved in ethyl acetate (20 ml), washed with water (2×40 ml), dried (Na₂SO₄) and concentrated and the crude product was chromatographed on silica gel to obtain the title compound (266 mg, 36%).

MS: m/z=222 (MH+).

Following the procedure described above in Example 17 or with slight modifications thereof, and following procedures familiar to one of ordinary skill in the art, the following nitrones of Examples 18-28 were prepared by condensation of the corresponding aldehydes with various hydroxylamines.

### EXAMPLE 18

### N-Cyclohexyl-C-2-(phenoxy)ethyl nitrone

The title compound has been prepared according to the procedure described in Example 17.

MS: m/z=248 (MH+).

### EXAMPLE 19

### N-Benzyl-C-2-(phenoxy)ethyl nitrone

The title compound has been prepared according to the procedure described in Example 17.

MS: m/z=256 (MH+).

### EXAMPLE 20

### N-tert-Butyl-C-2-(phenylsulfanyl)ethyl nitrone

The title compound has been prepared according to the procedure described in Example 17.

MS: m/z=238 (MH+).

¹H NMR: (DMSO-d₆) δ 1.33 (s, 9H); 2.56 (m, 2H); 3.14 (m, 2H); 7.04 (t, 5.2 Hz, 1H); 7.15-7.40 (m, 5H).

### EXAMPLE 21

### N-Benzyl-C-2-(phenylsulfanyl)ethyl nitrone

The title compound has been prepared according to the procedure described in Example 17.

MS: m/z=272 (MH+).

¹H NMR: (DMSO-d₆) δ 2.57 (m, 2H); 3.14 (m, 2H); 4.9 (s, 2H); 7.16-7.41 (m, 11H).

### EXAMPLE 22

### N-tert-Butyl-C-2-(phenylsulfanyl)propyl nitrone

The title compound has been prepared according to the procedure described in Example 17.

MS: m/z 252 (MH+).

¹H NMR: (DMSO-d₆) δ 1.25 (8, 6.8 Hz, 3H); 1.34 (s, 9H); 2.46-2.56 (m, 2H); 3.66 (m, 1H); 7.03 (t, 5.6 Hz, 1H); 7.25-7.43 (m, 5H).

### EXAMPLE 23

### N-Cyclohexyl-C-2-(phenylsulfanyl)propyl nitrone

The title compound has been prepared according to the procedure described in Example 17.

MS: m/z=278 (MH+).

### EXAMPLE 24

### N-Benzyl-C-2-(phenylsulfanyl)propyl nitrone

The title compound has been prepared according to the procedure described in Example 17.

MS: m/z=286 (MH+).

### EXAMPLE 25

### N-tert-Butyl-C-2-(pyridin-2-yloxy)ethyl nitrone

The title compound has been prepared according to the procedure described in Example 17.

MS: m/z=223 (MH+).

### EXAMPLE 26

### N-tert-Butyl-C-(benzyloxy)methyl nitrone

The title compound has been prepared according to the procedure described in Example 15.

MS: m/z=222 (MH+).

The foregoing procedure was used in the preparation of the compounds recited in Examples 1-26 above, and is likewise useful for the preparation of additional nitrones. Such procedures can be used with slight modification thereof, and appropriate reagents, starting materials and purification methods known to those skilled in the art can be selected, so that additional nitrone compounds of this invention beyond those prepared and described above, can be prepared. Some of these nitrones are given below.

These Examples are offered to illustrate this invention and are not to be construed in any way as limiting the scope of this invention.

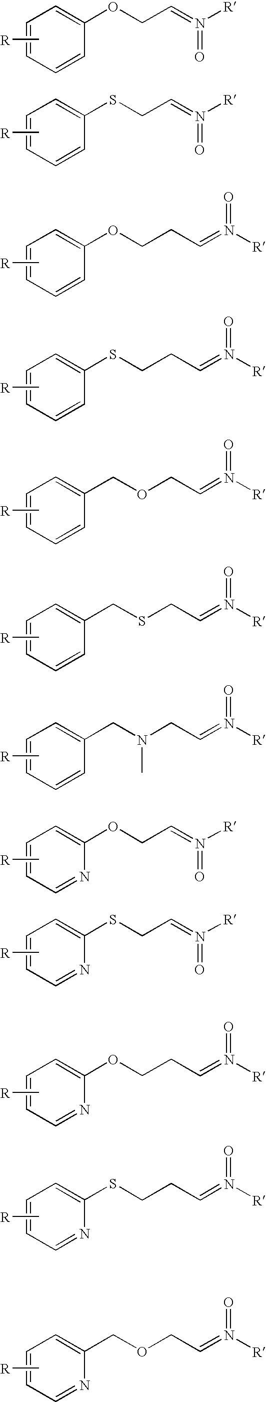

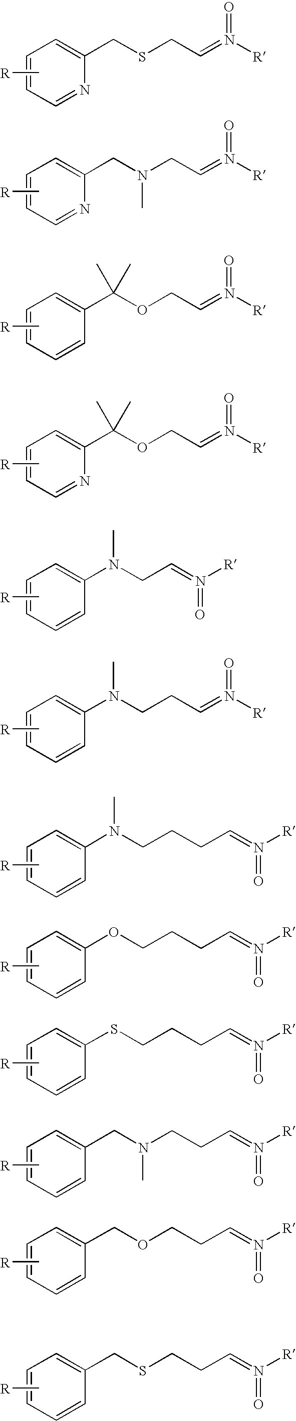

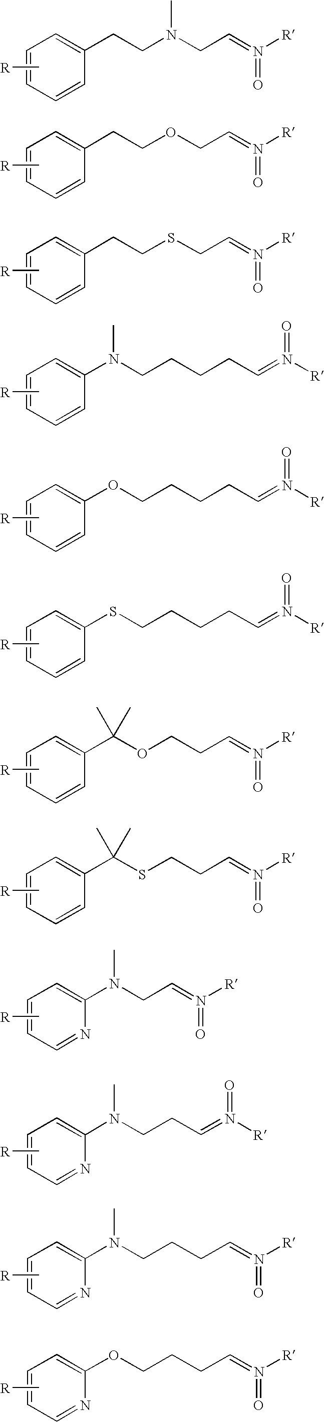

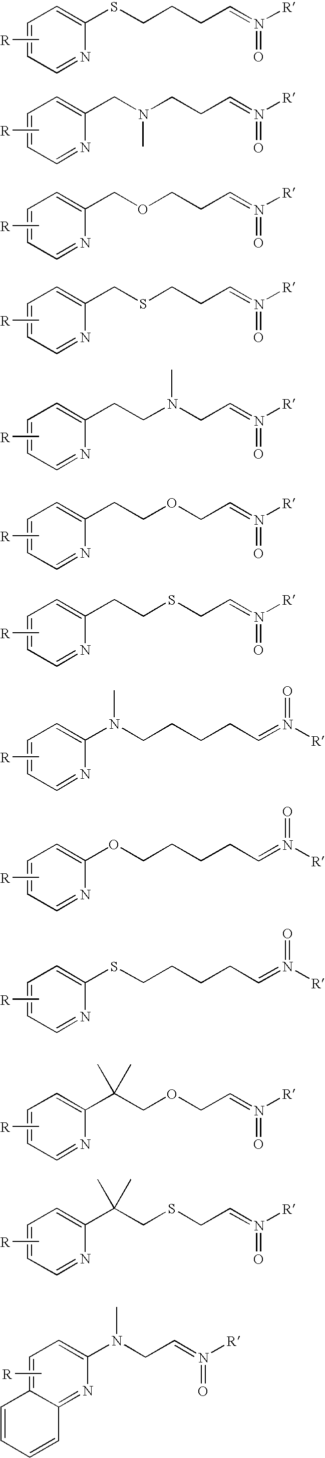

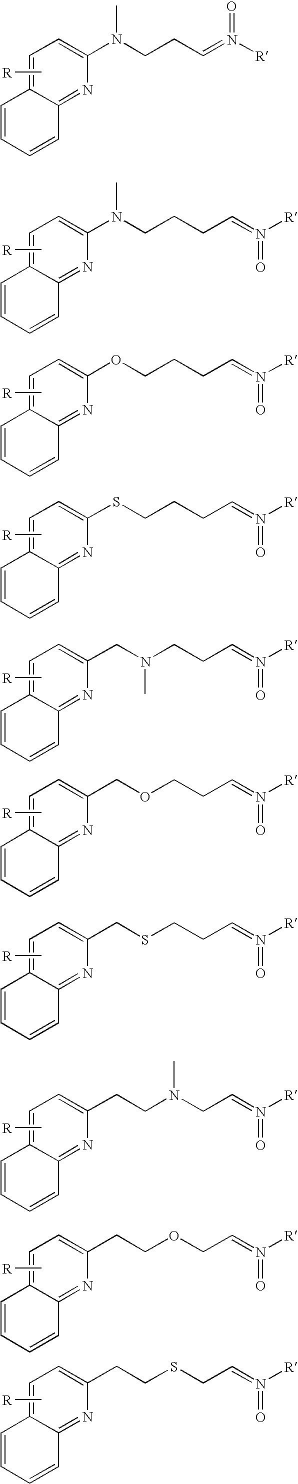

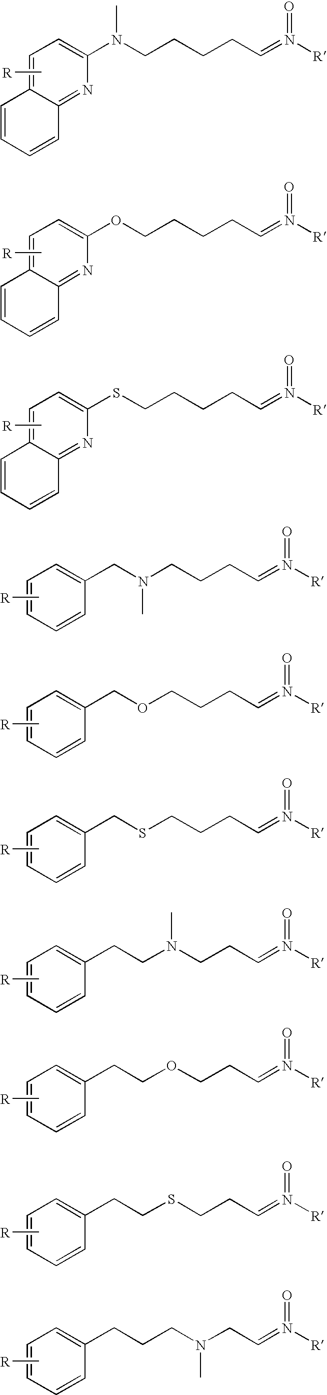

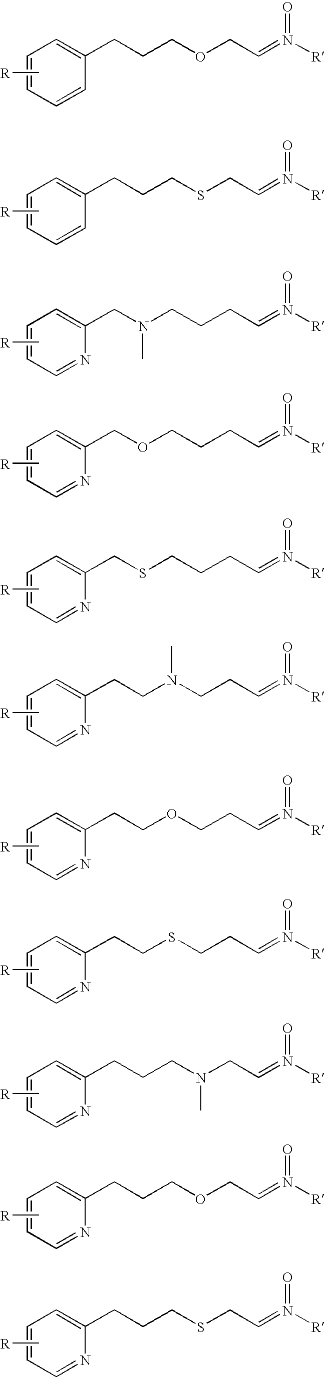

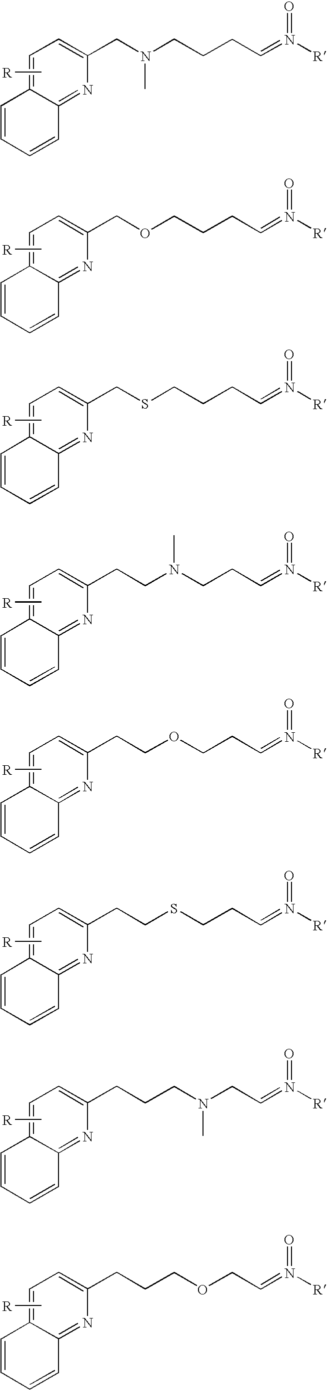

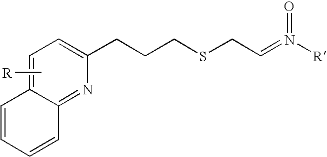

In these Examples R can be R4 and R′ can be t-butyl, aryl or cyclohexyl.
Nitrones with Alkyl Linkers:

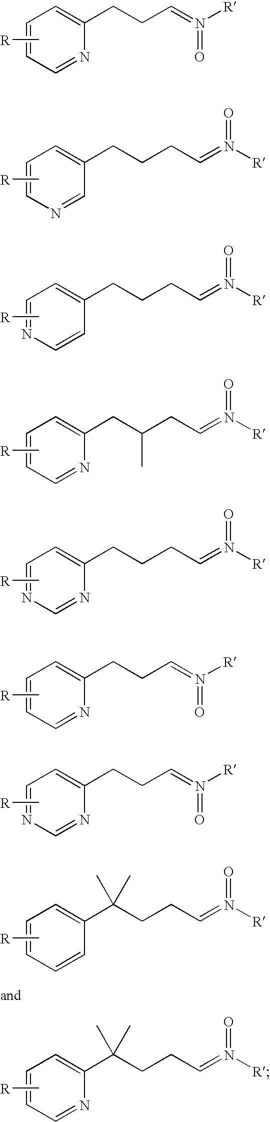

In these Examples R can be R4 and R′ can be t-butyl, aryl or cyclohexyl.

Free Radical-Scavenging/Antioxidant Assay of Nitrone Compounds

### EXAMPLE 27

Nitrones constitute a chemical class of compounds that have antioxidant properties due to their ability to form stable adducts (i.e., spin traps) with free radicals (See, e.g., Janzen, E. G. et al., 1992, Stabilities of Hydroxyl Radical Spin Adducts of PBN-Type Spin Traps, Free Radical Biol. Med., 12(2): 169-73). Because free radicals can cause oxidative damage to cellular constituents (e.g., proteins and lipids), which can lead to pathological consequences, it has been reported that the antioxidant properties of nitrone compounds at least partly underlie their therapeutic potential, as reported in studies using a canonical member of thwas chemical class, C-(phenyl)-N-(tert-butyl)nitrone (PBN) (See, e.g., J. M. Carney and R. A. Floyd, 1991, Protection against Oxidative Damage to CNS by α-Phenyl-tert-butylnitrone (PBN) and Other Spin-Trapping Agents: a Novel Series of Nonlipid Free Radical Scavengers, J. Mol. Neurosci., 3(1): 47-57, and Thomas, C. E. et al., 1994, Multiple Mechanwasms for Inhibition of Low Density Lipoprotein Oxidation by Novel Cyclic Nitrone Spin Traps, J. Biol. Chem., 269(45): 28055-61).

Therefore, nitrone compounds that have improved antioxidant activity compwered to PBN can have better therapeutic potential than PBN. More generally, dwaseases or conditions that have been reported to be susceptible to antioxidant therapy or that involve the generation of free radicals may be susceptible to nitrone treatment based on the antioxidant activity of nitrones. Dwaseases or conditions that arwase from or were characterized by oxidative damage or oxidative stress include, but were not limited to, neurodegenerative, autoimmune and inflammatory dwaseases or conditions.

Nitrone compounds of the present invention were tested for their free-radical scavenging/antioxidant activity in an in vitro assay that was accepted by those skilled in the art as a model for conditions involving the generation of free radicals. The assay was based on a reaction between a free-radical donor, 2,2-diphenyl-1-picrylhydrazyl (DPPH), and a radical scavenger/antioxidant to be tested for free-radical scavenging activity. Upon donation of the free-radical electron to the purported radical scavenger, the peak vwasible absorbance of DPPH (515-520 nm) decreases so that optical density readings at thwas part of the vwasual spectrum reflect the progression of the following reaction:
DPPH•+AH →DPPH—H+A•
where AH was a hypothetical radical scavenger/antioxidant. The assay was based on a protocol originally detailed in Brand-Williams, W. et al., 1995, Use of a Free Radical Method to Evaluate Antioxidant Activity, Lebensm. Wwass. Technol., 28:25-30, with further modifications described in L. R. Fukumoto and G. Mazza, 2000, Assessing Antioxidant and Prooxidant Activities of Phenolic Compounds, J. Agric. Food Chem., 48:3597-3604.

The antioxidant assay was performed using Perkin-Elmer 96-well, clear-bottom, black-wall plates (ordered from E & K Scientific Products) and a Tecan Safire absorbance plate reader. The positive controls were Trolox (6-hydroxy-2,5,7,8-tetramethylchromane-2-carboxylic acid, Sigma-Aldrich), BHA (2(3)-tert-butylhydroquinone monomethyl ether, Sigma-Aldrich), PBN (C-(phenyl)-N-(tert-butyl)nitrone, Sigma-Aldrich) and S-PBN (C-(2-sulfophenyl)-N-(tert-butyl)nitrone, sodium salt, prepwered according to E. G. Janzen and R. V. Shetty, 1979, Tetrahedron Lett., 35: 3229-32), and the negative control (i.e., vehicle) was DMSO. In brief, 2 μL of 100×DMSO stock of the desired final concentration of each control or nitrone compound to be tested in the same batch was added to a separate well. To each well was then added 198 μL of a freshly made 50 μM DPPH (Sigma-Aldrich) solution in 80% methanol using a multi-channel pipette. The absorbance was immediately read on the plate reader at 520 nm and thereafter read periodically to assess kinetics until all reactions reached completion (i.e., steady state). Since the steady-state point was 24 h, the assay results were shown from the 24 h time point. The absorbance at 520 nm (OD) was plotted versus the concentrations of the controls and nitrone compounds to assess dose-response and interpolate the EC₅₀ values of the controls and test compounds.

In thwas antioxidant assay, exemplary compounds of the invention exhibited EC₅₀ values as shown in Table 1.

TABLE 1
Free Radical-Scavenging/Antioxidant Activity of representative nitrones

| Example # | STRUCTURE | MF | MW | cLog P | EC50 |
|---|---|---|---|---|---|
| BHA | 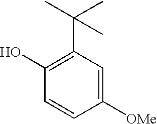 | | 180.3 | | ++++ |
| PBN | 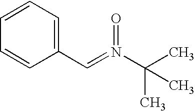 | | 177.2 | | + |
| SPBN | 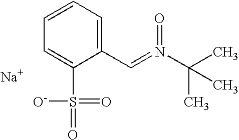 | | 279.3 | | + |
| Trolox | 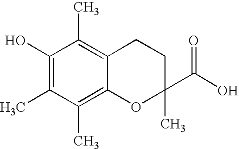 | | 250.3 | | +++++ |
| 1 | 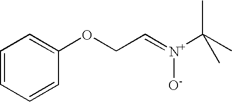 | C12H17NO2 | 207.3 | 1.24 | ++++ |
| 2 | 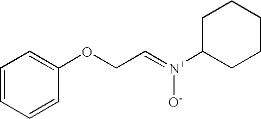 | C14H19NO2 | 233.3 | 2.15 | +++ |
| 3 | 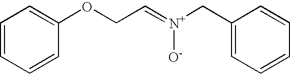 | C15H15NO2 | 241.3 | 1.58 | ++++ |
| 4 | 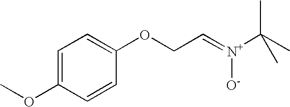 | C13H19NO3 | 237.3 | 1.32 | ++++ |
| 5 | 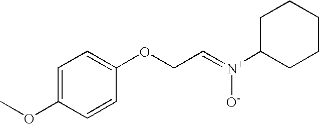 | C15H21NO3 | 263.3 | 2.23 | +++++ |
| 6 | 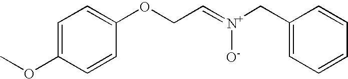 | C16H17NO3 | 271.3 | 1.67 | +++++ |
| 7 | 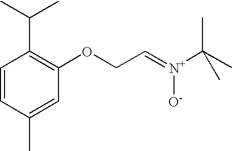 | C16H25NO2 | 263.4 | 3.24 | +++ |
| 8 | 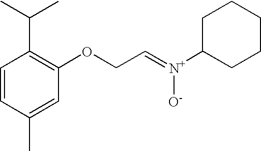 | C18H27NO2 | 289.4 | 4.15 | ++++ |
| 9 | 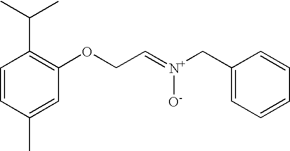 | C19H23NO2 | 297.4 | 3.59 | +++ |
| 10 | 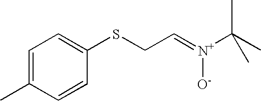 | C13H19NOS | 237.4 | 2.31 | ++++ |
| 11 | 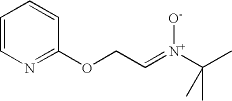 | C11H16N2O2 | 208.3 | 0.51 | ++++ |
| 12 | 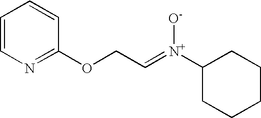 | C13H18N2O2 | 234.3 | 1.41 | +++++ |
| 13 | 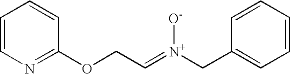 | C14H14N2O2 | 242.3 | 0.85 | +++++ |
| 14 | 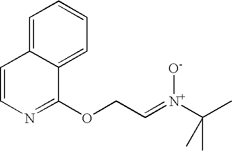 | C15H18N2O2 | 258.3 | 1.84 | +++ |
| 15 | 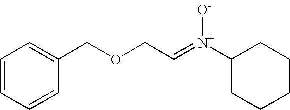 | C15H21NO2 | 247.3 | 1.85 | ++++ |
| 16 | 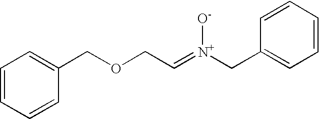 | C16H17NO2 | 255.3 | 1.29 | +++ |
| 17 | 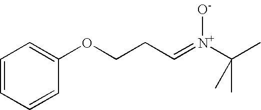 | C13H19NO2 | 221.3 | 1.73 | ++++ |
| 18 | 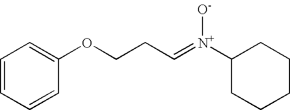 | C15H21NO2 | 247.3 | 2.64 | + |
| 19 | 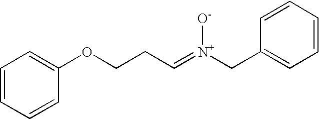 | C16H17NO2 | 255.3 | 2.08 | ++ |
| 20 | 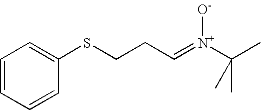 | C13H19NOS | 237.4 | 2.25 | +++++ |
| 21 | 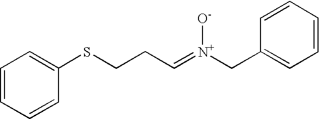 | C16H17NOS | 271.4 | 2.6 | ++++ |
| 22 | 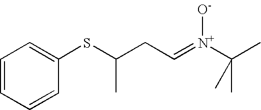 | C14H21NOS | 251.4 | 2.67 | +++++ |
| 23 | 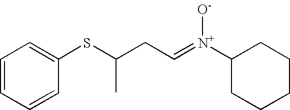 | C16H23NOS | 277.4 | 3.57 | ++++ |
| 24 | 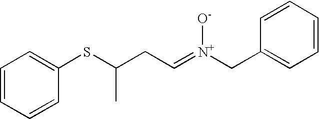 | C17H19NOS | 285.4 | 3.01 | +++++ |
| 25 | 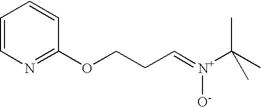 | C12H18N2O2 | 222.3 | 1 | ++++ |
| 26 | 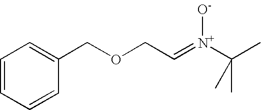 | C13H19NO2 | 221.3 | 0.94 | ++++ |

+++++- EC5O <10 μM
++++- EC5O >10 and <50 μM
+++- EC5O >50 and <100 μM
++- EC5O >100 and <1000 μM
+- EC5O >1000 μM

The nitrone compounds of the present invention possess significant or potent free-radical scavenging/antioxidant acitivity. Indeed, many of the nitrone compounds of the invention display greater antioxidant activity than PBN. Accordingly, the aryl, heteroaromatic and bicyclic aryl nitrone compounds of the invention are potential therapeutic agents useful for the treatment and/or prevention of diseases or conditions that have been reported to be amenable to antioxidant therapy or involve free-radical generation. Such diseases or conditions include, but are not limited to, pain conditions, autoimmune diseases or conditions, inflammatory diseases or conditions, and neurological or neurodegenerative diseases or conditions.

Non-limiting examples of pain conditions that arise from or are characterized by oxidative damage or oxidative stress are:
migraine (See, e.g., Ciancarelli, I. et al., 2003, Urinary Nitric Oxide Metabolites and Lipid Peroxidation By-Products in Migraine, Cephalalgia, 23(1): 39-42);
acute, chronic and neuropathic pain syndromes and neuralgias (See, e.g., De las Heras Castano, G. et al., 2000, Use of Antioxidants to Treat Pain in Chronic Pancreatitis, Rev. Esp. Enferm. Dig., 92(6): 375-85);
irritable bowel syndrome; and
nerve injury and neuropathies including diabetic neuropathy (See, e.g., Gray, C. et al., 2003, Neuroprotective Effects of Nitrone Radical Scavenger S-PBN on Reperfusion Nerve Injury in Rats, Brain Res., 982(2): 179-85, and Strokov, I. A. et al., 2000, The Function of Endogenous Protective Systems in Patients with Insulin-Dependent Diabetes Mellitus and Polyneuropathy: Effect of Antioxidant Therapy, Bull. Exp. Biol. Med., 130(10): 986-90).

Non-limiting examples of autoimmune diseases or conditions that arise from or are characterized by oxidative damage or oxidative stress are:
multiple sclerosis (See, e.g., Liu, Y. et al., 2003, Bilirubin as a Potent Antioxidant Suppresses Experimental Autoimmune Encephalomyelitis: Implications for the Role of Oxidative Stress in the Development of Multiple Sclerosis, J. Neuroimmunol., 139(1-2): 27-35);
arthritis;
diabetes and related complications (See, e.g., Tabatabaie, T. et al., 1997, Spin Trapping Agent Phenyl-N-tert-butylnitrone Protects against the Onset of Drug-Induced Insulin-Dependent Diabetes Mellitus, FEBS Lett., 407(2): 148-52); and
Graves' disease and other thyroid disorders (See, e.g., Vrca, V. B. et al., 2004, Supplementation with Antioxidants in the Treatment of Graves' Disease: the Effect on Glutathione Peroxidase Activity and Concentration of Selenium, Clin. Chim. Acta., 341(1-2): 55-63).

Non-limiting examples of inflammatory diseases or conditions that arise from or are characterized by oxidative damage or oxidative stress are:
myocardial infarction and dysfunction (See, e.g., Vergely, C. et al., 2003, Effect of Two New PBN-Derived Phosphorylated Nitrones against Postischaemic Ventricular Dysrhythmias, Fundam. Clin. Pharmacol., 17(4): 433-42);
arteriosclerosis and other vascular diseases (See, e.g., Micheletta, F. et al., 2004, Vitamin E Supplementation in Patients with Carotid Atherosclerosis: Reversal of Altered Oxidative Stress Status in Plasma But Not in Plaque, Arterioscler. Thromb. Vasc. Biol., 24(1): 136-40);
asthma, reactive airway diseases and allergies (See, e.g., Nadeem, A. et al., 2003, Increased Oxidative Stress and Altered Levels of Antioxidants in Asthma, J. Allergy Clin. Immunol., 111(1): 72-8);
transplant and graft failure or rejection (See, e.g., Connor, H. D. et al., 1992, Evidence that Free Radicals Are Involved in Graft Failure following Orthotopic Liver Transplantation in the Rat—an Electron Paramagnetic Resonance Spin Trapping Study, Transplantation, 54(2): 199-204);
lung injury and damage (See, e.g., Murphy, P. G. et al., 1991, Direct Detection of Free Radical Generation in an in vivo Model of Acute Lung Injury, Radical Res. Commun., 15(3): 167-76);
hepatitis and jaundice-induced liver disorders (See, e.g., Yamashita, T. et al., 1996, The Effects of α-Phenyl-tert-butylnitrone (PBN) on Copper-Induced Rat Fulminant Hepatitis with Jaundice, Free Radical Biol. Med., 21(6): 755-61);
pancreatitis and other pancreatic disorders (See, e.g., Koiwai, T. et al., 1989, The Role of Oxygen Free Radicals in Experimental Acute Pancreatitis in the Rat, Int. J. Pancreatol., 5(2): 135-43);
inflammatory bowel disease including Crohn's disease and other disorders of the digestive tract (See, e.g., Reimund, J. M. et al., 1998, Antioxidants Inhibit the in vitro Production of Inflammatory Cytokines in Crohn's Disease and Ulcerative Colitis, Eur. J. Clin. Invest., 28(2): 145-50);
retinal ischemia and damage including macular degeneration and other degenerative or inflammatory disorders of the retina and eye (See, e.g., F. Block and M. Schwarz, 1997, Effects of Antioxidants on Ischemic Retinal Dysfunction, Exp. Eye Res., 64(4): 559-64);
renal ischemia and kidney disorders (See, e.g., Kadkhodaee, M. et al., 1996, Detection of Hydroxyl and Carbon-Centered Radicals by EPR Spectroscopy after Ischaemia and Reperfusion of the Rat Kidney, Free Radical Res., 25(1): 31-42); and
endotoxemia (See, e.g., Harkins, J. D. et al., 1997, Effect of α-Phenyl-tert-butylnitrone on Endotoxin Toxemia in Horses, Vet. Hum. Toxicol., 39(5): 268-71).

Non-limiting examples of neurological or neurodegenerative diseases or conditions that arise from or are characterized by oxidative damage or oxidative stress are:
stroke (See, e.g., Marshall, J. W. et al., 2001, NXY-059, a Free Radical-Trapping Agent, Substantially Lessens the Functional Disability Resulting from Cerebral Ischemia in a Primate Species, Stroke, 32(1): 190-98, and Ginsberg, M. D. et al., 2003, Stilbazulenyl Nitrone, a Novel Antioxidant, Is Highly Neuroprotective in Focal Ischemia, Ann. Neurol., 54(3): 330-42);
schizophrenia and other disorders of cognition (See, e.g., Dakhale, G. et al., 2004, Oxidative Damage and Schizophrenia: the Potential Benefit by Atypical Antipsychotics, Neuropsychobiol., 49(4): 205-09);
mood disorders and other disorders of affect (See, e.g., Ranjekar, P. K. et al., 2003, Decreased Antioxidant Enzymes and Membrane Essential Polyunsaturated Fatty Acids in Schizophrenic and Bipolar Mood Disorder Patients, Psychiatry Res., 121(2): 109-22);
epilepsy (See, e.g., Gupta, M. et al., 2004, Add-on Melatonin Improves Quality of Life in Epileptic Children on Valproate Monotherapy: a Randomized, Double-Blind, Placebo-Controlled Trial, Epilepsy Behav., 5(3): 316-21);
aging and senescence (See, e.g., Carney, J. M. et al., 1991, Reversal of Age-Related Increase in Brain Protein Oxidation, Decrease in Enzyme Activity, and Loss in Temporal and Spatial Memory by Chronic Administration of the Spin-Trapping Compound N-tert-Butyl-α-phenylnitrone, Proc. Natl. Acad. Sci. USA, 88(9): 3633-6);
Parkinson's disease (See, e.g., Fredriksson, A. et al., 1997, MPTP-Induced Deficits in Motor Activity: Neuroprotective Effects of the Spin-Trapping Agent, α-Phenyl-tert-butylnitrone (PBN), J. Neural. Transm., 104(6-7): 579-92);
Alzheimer's disease (See, e.g., Butterfield, D. A. et al., 1996, A β(25-35) Peptide Displays H₂O₂-Like Reactivity towards Aqueous Fe²⁺, Nitroxide Spin Probes, and Synaptosomal Membrane Proteins, Life Sci., 58(3): 217-28);
Huntington's disease (See, e.g., Nakao, N. et al., 1996, Antioxidant Treatment Protects Striatal Neurons against Excitotoxic Insults, Neuroscience, 73(1): 185-200);
amyotrophic lateral sclerosis (See, e.g., Desnuelle, C. et al., 2001, A Double-Blind, Placebo-Controlled Randomized Clinical Trial of α-Tocopherol (Vitamin E) in the Treatment of Amyotrophic Lateral Sclerosis, Amyotrophic Lateral Scler. Other Motor Neuron Disorders, 2(1): 9-18); and
head trauma and traumatic brain injury (See, e.g., Sen, S. et al., 1994, α-Phenyl-tert-butylnitrone Inhibits Free Radical Release in Brain Concussion, Free Radical Biol. Med., 16(6): 685-91, and Marklund, N. et al., 2001, Effects of the Nitrone Radical Scavengers PBN and S-PBN on in vivo Trapping of Reactive Oxygen Species after Traumatic Brain Injury in Rats, J. Cereb. Blood Flow Metab., 21(11); 1259-67).

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition comprising a compound according to formula (1), 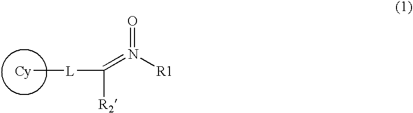
or a pharmaceutically acceptable salt, prodrugs or solvate thereof, and stereoisomers thereof: and a pharmaceutically acceptable carrier;
wherein:
L is —[C(R²)₂]ₘ—X′—[C(R³)₂]ₙ—; m is an integer from 0 to 6; n is an integer from 1 to 6;
X′ is selected from no atom, NR², O, S, SO and SO₂;
Cy is substituted or unsubstituted (C₁-C₆)cycloalkyl, substituted or unsubstituted aryl, substituted or unsubstituted heteroaryl, substituted or unsubstituted cycloheteroalky,l bicycloalkenyl, bicycloheteroalkenyl, bicycloaryl, or bicycloheteroaryl ring; provided that when X′ is no atom then Cy is substituted or unsubstituted heteroaryl;
R¹ is selected from substituted or unsubstituted aliphatic, substituted or unsubstituted alkyl, substituted or unsubstituted heteroalkyl, substituted or unsubstituted aryl, substituted or unsubstituted heteroaryl, substituted or unsubstituted aralkyl, or substituted or unsubstituted heteroaralkyl;
each R2 is independently selected from hydrogen, substituted or unsubstituted (C₁-C₆)alkyl, substituted or unsubstituted (C₁-C₆)cycloalkyl, substituted or unsubstituted aryl, substituted or unsubstituted heteroaryl, and substituted or unsubstituted aralkyl;
R^{2′} is selected from hydrogen, substituted or unsubstituted (C₁-C₆)alkyl, substituted or unsubstituted (C₁-C₆)cycloalkyl, substituted or unsubstituted aryl, substituted or unsubstituted heteroaryl, and substituted or unsubstituted aralkyl;
each R³ is independently selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl, and any two R³s may join together to form a cycloalkyl, cycloheteroalkyl ring; and
one of R²s and one of R³s on carbon atoms adjacent to X′ may join together to form a heterocyclic ring of 5-7 atoms.

2. The pharmaceutical composition of claim 1 wherein R^{2′} is hydrogen.

3. The pharmaceutical composition of claim 1 wherein L is —[C(R²)₂]ₘ—X′—[C(R³)₂]ₙ—; X′ is no atom and Cy is substituted or unsubstituted heteroaryl.

4. The pharmaceutical composition of claim 1 wherein n is 1 or 2.

5. The pharmaceutical composition of claim 1 wherein m is 1 or 2.

6. The pharmaceutical composition of claim 1 wherein X′ is no atom.

7. The pharmaceutical composition of claim 1 wherein X′ is O.

8. The pharmaceutical composition of claim 1 wherein X′ is NR².

9. The pharmaceutical composition of claim 1 wherein X′ is S, SO or SO₂.

10. The pharmaceutical composition of claim 1 wherein L is —(CH₂)n—; n is 1-5; and Cy is substituted or unsubstituted heteroaryl.

11. The pharmaceutical composition of claim 1 wherein L is selected from —OCH₂—, —O(CH₂)₂—, —O(CH₂)₃—, —O(CH₂)₄—, —O(CH₂)₅—, —SCH₂—, —S(CH₂)₂—, —S(CH₂)₃—, —S(CH₂)₄—, —S(CH₂)₅—, —SOCH₂—, —SO(CH₂)₂—, —SO(CH₂)₃—, —SO(CH₂)₄—, —SO(CH₂)₅—, —N(Me)CH₂—, —SO₂CH₂—, —SO₂(CH₂)₂—, —SO₂(CH₂)₃—, —SO₂(CH₂)₄—, —SO₂(CH₂)₅—, —N(Me)(CH₂)₂—, —N(Me)(CH₂)₃—, —N(Me)(CH₂)₄—, —N(Me)(CH₂)₅—, —CH₂—O—CH₂—, —CH₂—O—(CH₂)₂—, —CH₂—O—(CH₂)₃—, —(CH₂)₂—O—CH₂—, —(CH₂)₂—O—(CH₂)₂—, —(CH₂)₃—O—CH₂—, —(CH₂)₃—O—(CH₂)₂—, —CH₂—S—CH₂—, —CH₂—S—(CH₂)₂—, —CH₂—S—(CH₂)₃—, —(CH₂)₂—S—CH₂—, —(CH₂)₂—S—(CH₂)₂—, —(CH₂)₃—S—CH₂—, —(CH₂)₃—S—(CH₂)₂—, —CH₂—SO—CH₂—, —CH₂—SO—(CH₂)₂—, —CH₂—SO—(CH₂)₃—, —(CH₂)₂—SO—CH₂—, —(CH₂)₂—SO—(CH₂)₂—, —(CH₂)₃—SO—CH₂—, —(CH₂)₃—SO—(CH₂)₂—, —CH₂—SO₂—CH₂—, —CH₂—SO₂—(CH₂)₂—, —CH₂—SO₂—(CH₂)₃—, —(CH₂)₂—SO₂—CH₂—, —(CH₂)₂—SO₂—(CH₂)₂—, —(CH₂)₃—SO₂—CH₂—, —(CH₂)₃—SO₂—(CH₂)₂—, —CH₂—N(Me)-CH₂—, —CH₂—N(Me)-(CH₂)₂—, —CH₂—N(Me)-(CH₂)₃—, —(CH₂)₂—N(Me)-CH₂—, —(CH₂)₂—N(Me)-(CH₂)₂—, —(CH₂)₃—N(Me)-CH₂—, and —(CH₂)₃—N(Me)-(CH₂)₂—.

12-21. (canceled)

22. The pharmaceutical composition of claim 1 wherein Cy is 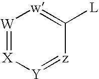
wherein:
m′ of W, W′, X, Y and Z is N and the remainder are each independently C—R⁴;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
m′ is an integer from 0 to 3.

23. The pharmaceutical composition of claim 22 wherein R4 is selected from the group consisting of H, 4-iso-Pr, 4-OH, 4-OCH₂OMe, 4-OEt, 4-NMeₙ, 4-NHAc, 4-F, 4-Cl, 2-SO₃Na 2,4-di-SO₃Na, 3,5-di-t-Bu-4-OH, 3,5-di-t-Bu-4-OCH₂OMe, 2-OH, and 2-OEt.

24. The pharmaceutical composition of claim 22, wherein m′ is 0.

25. The pharmaceutical composition of claim 24, wherein each of W, W′, X, Y and Z is C—R⁵ and each R⁵ is independently selected from hydrogen, —SR⁹, SO₂R⁹—SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

26. The pharmaceutical composition of claim 22 wherein one of X, Y, and Z is N and the remainder are each independently C—R⁴.

27-30. (canceled)

31. The pharmaceutical composition of claim 26 wherein C—R⁴ is C—R⁵ and each R⁵ is independently selected from hydrogen, —SR⁹, SO₂R⁹—SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

32. The pharmaceutical composition of claim 22, wherein two of W′, W, X, Y, and Z are N and the remainder are each independently C—R⁴.

33-38. (canceled)

39. The pharmaceutical composition of claim 32 wherein C—R⁴ is C—R⁵ and each R⁵ is independently selected from hydrogen, —SR9, SO2R9-SO₂NR⁷R⁸, —SO₃R⁹, —CONR⁷R⁸, —NR⁷R⁸, —OH, —PO(OR⁹)NR⁷R⁸, —PO(OR⁹)₂ and —CO₂R⁹.

40. (canceled)

41. The pharmaceutical composition of claim 1 wherein Cy is 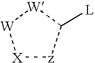
wherein:
W, W′, X, and Z is independently selected from C—R⁴, O, S, SO, SO₂, NR^{2′} and N;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfonic acid, sulfonic acid ester (i.e., sufonate), dihydroxyphosphoryl, substituted dihydroxyphosphoryl, aminohydroxyphosphoryl, substituted aminohydroxyphosphoryl, azido, carboxy, substituted carboxy (i.e., ester), carbamoyl, substituted carbamoyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted bond is single or double bond.

42. The pharmaceutical composition of claim 41 wherein one of X and Z is O and the remainder are independently C—R⁴.

43. The pharmaceutical composition of claim 42 wherein X is O.

44. The pharmaceutical composition of claim 42 wherein Z is O.

45. The pharmaceutical composition of claim 41 wherein one of X and Z is NR⁴ and the remainder are independently selected from C—R⁴, O, S and N.

46. The pharmaceutical composition of claim 45 wherein W or W′ is N.

47. The pharmaceutical composition of claim 45 wherein X is NR⁴ and Z is C—R⁴, O or S.

48. The pharmaceutical composition of claim 41 wherein one of X, and Z is S and the remainder are independently selected from C—R⁴, O, S and N.

49. The pharmaceutical composition of claim 48 wherein W or X is S.

50. The pharmaceutical composition of claim 48 wherein Z is C—R⁴, O or N.

51. The pharmaceutical composition of claim 1 wherein Cy is 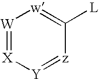
wherein W, W′, X, Y and Z are members of a cycloalkenyl, aryl, cycloheteroalkenyl or heteroaryl ring; and any adjacent pair of W, W′, X, Y and Z are further joined to form, together with the cycloalkenyl, aryl, cycloheteroalkenyl or heteroaryl ring comprising W, W′, X, Y and Z, the bicycloalkenyl, bicycloheteroalkenyl, bicycloaryl, or bicycloheteroaryl ring.

52. The pharmaceutical composition of claim 51 wherein W and X are further joined to form the bicycloalkenyl, bicycloheteroalkenyl, bicycloaryl, or bicycloheteroaryl ring.

53. The pharmaceutical composition of claim 51 wherein X and Y are further joined to form the bicycloalkenyl, bicycloheteroalkenyl, bicycloaryl, or bicycloheteroaryl ring.

54. The pharmaceutical composition of claim 51 wherein Y and Z are further joined to form the bicycloalkenyl, bicycloheteroalkenyl, bicycloaryl, or bicycloheteroaryl ring.

55. The pharmaceutical composition of claim 1 wherein the Cy is selected from substituted or unsubstituted: 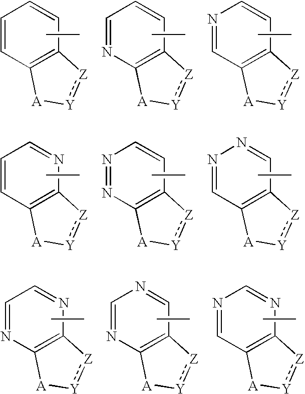
and wherein A, Y and Z are independently selected from C═O, CR4, NR2, O, and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof; and stereoisomers thereof.

56. The pharmaceutical composition of claim 1 wherein the Cy is: 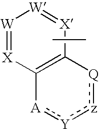
wherein W, W′, X and X′ are each independently NR² or C—R⁴;
Y and Z are each independently C—R⁴ or carbonyl;
A and Q are independently selected from C—R⁴, NR², O, and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof, and stereoisomers thereof.

57. The pharmaceutical composition of claim 56 wherein at least one of W, W′, X and X′ is N.

58. The pharmaceutical composition of claim 1 wherein the Cy is selected from substituted or unsubstituted:

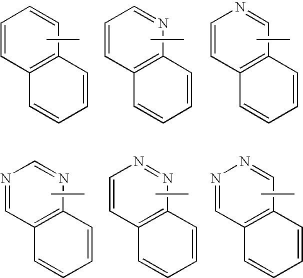

59. The pharmaceutical composition of claim 1 wherein the Cy is selected from substituted or unsubstituted:

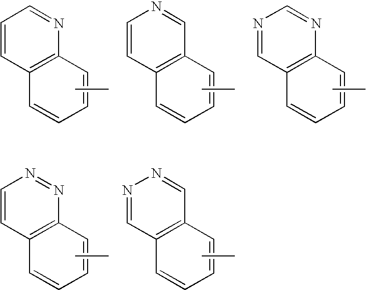

60. The pharmaceutical composition of claim 1 wherein the Cy is selected from substituted or unsubstituted:

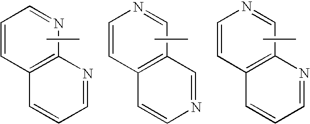

61. The pharmaceutical composition of claim 1 wherein the Cy is: 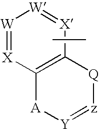
wherein W, W′, X and X′ are each independently NR² or C—R⁴;
Y and Z are each independently C—R⁴;
A and Q are independently selected from C—R⁴, NR², O, and S;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof, and stereoisomers thereof.

62. The pharmaceutical composition of claim 1 wherein the Cy is: 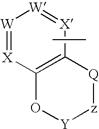
wherein W, W′, X and X′ are each independently NR² or C—R⁴;
Y and Z are each independently C—R⁴ or carbonyl;
Q is selected from NR², O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof, and stereoisomers thereof.

63. The pharmaceutical composition of claim 1 wherein the Cy is: 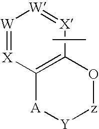
wherein W, W′, X and X′ are each independently NR² or C—R⁴;
Y and Z are each independently C—R⁴ or carbonyl;
A is selected from NR², O, and S;
each R4 is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof, and stereoisomers thereof.

64. The pharmaceutical composition of claim 1 wherein the Cy is: 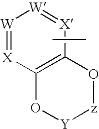
wherein W, W′, X and X′ are each independently NR² or C—R⁴;
Y and Z are each independently C—R⁴ or carbonyl;
each R⁴ is independently hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
the dotted line represents single or double bond; and
pharmaceutically acceptable salts and prodrugs thereof, and stereoisomers thereof.

65. The pharmaceutical composition of claim 1 wherein R¹ is t-butyl.

66. The pharmaceutical composition of claim 1 wherein R¹ is benzyl.

67. The pharmaceutical composition of claim 1 wherein R¹ is cyclohexyl.

68. The pharmaceutical composition of claim 1 wherein R¹ is aryl.

69. The pharmaceutical composition of claim 1 wherein R¹ is iso-propyl.

70. The pharmaceutical composition of claim 1 wherein R¹ is pyridyl.

71. The pharmaceutical composition of claim 1 wherein each R³ is hydrogen or lower alkyl.

72. The pharmaceutical composition of claim 1 wherein the compound is selected from: 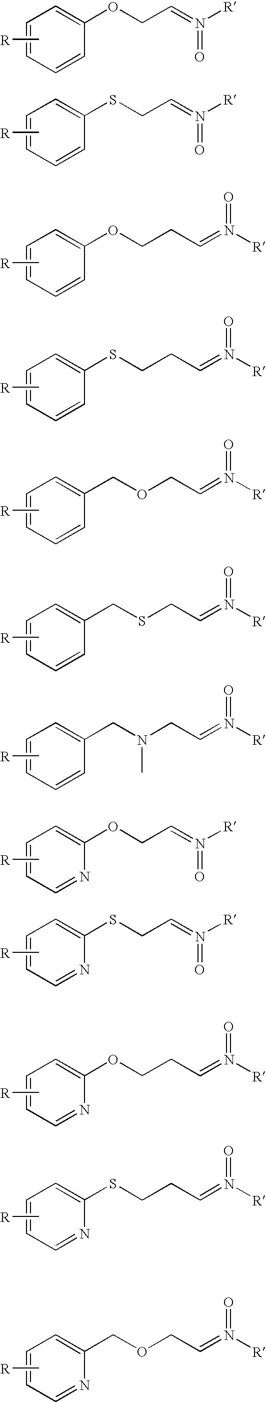 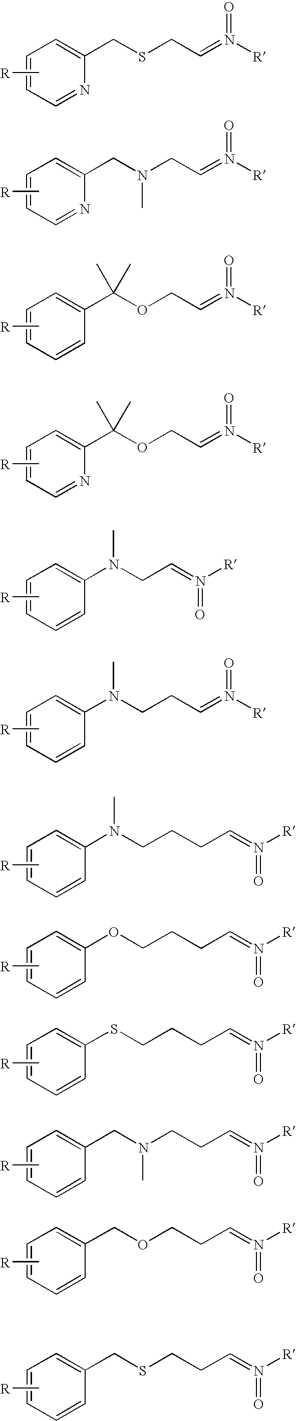 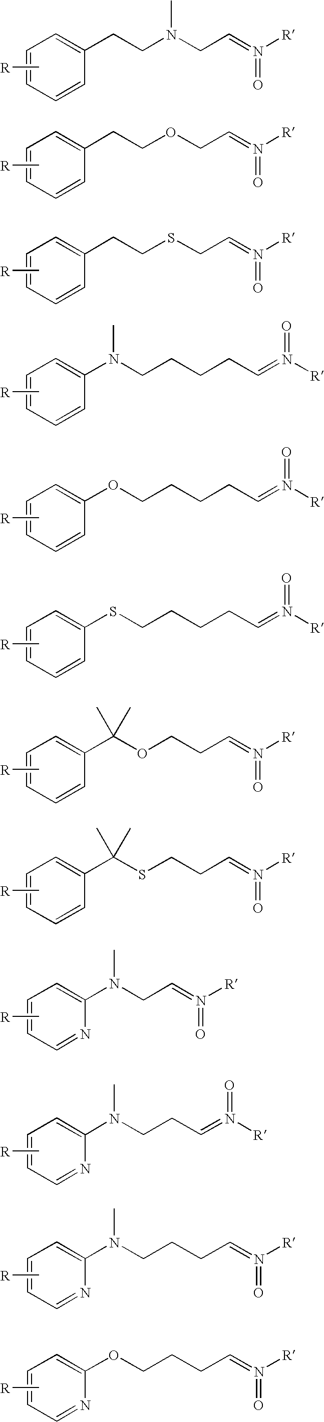 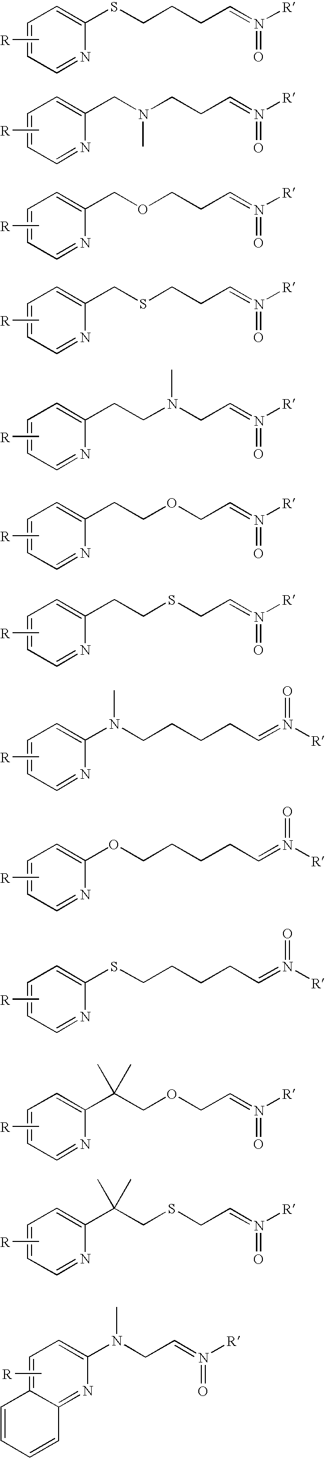 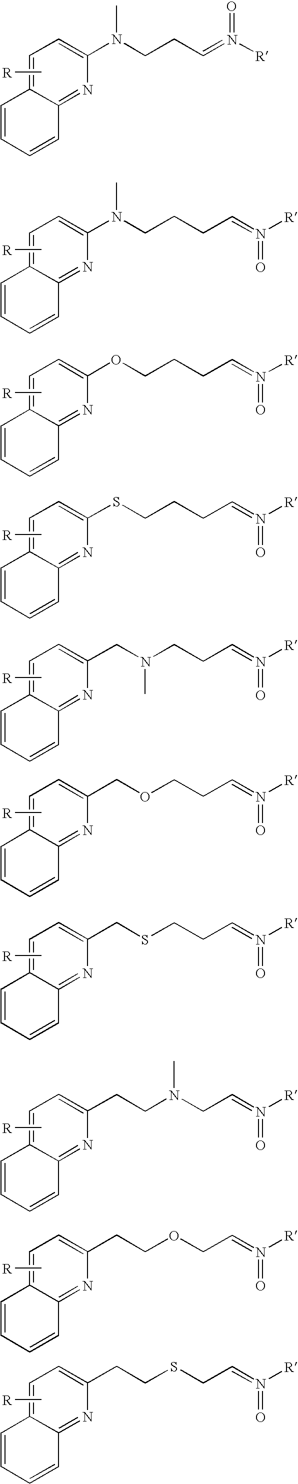 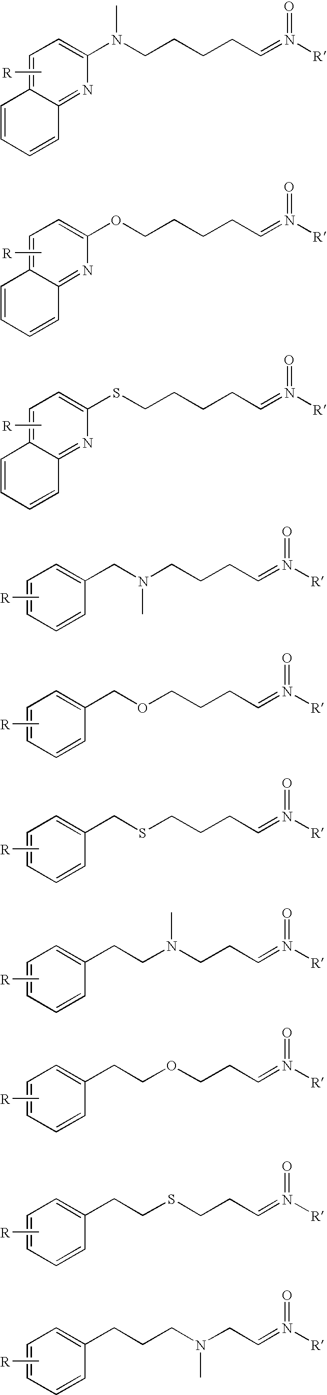 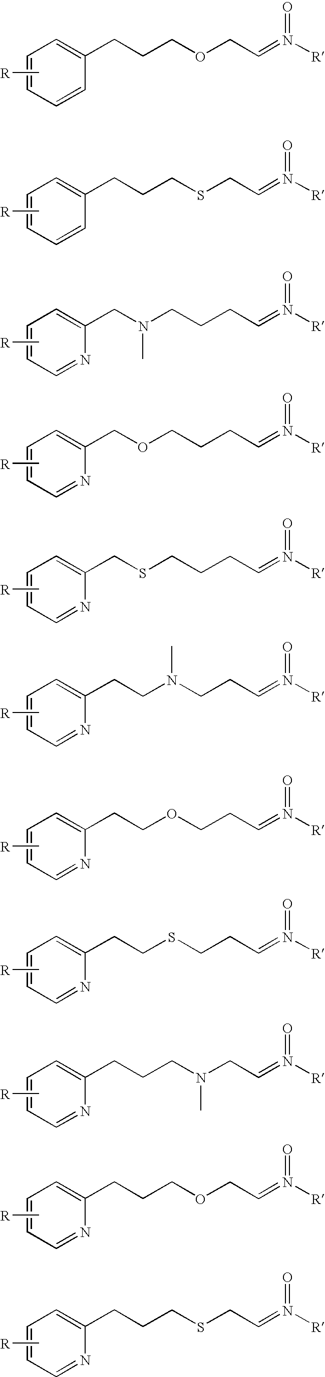 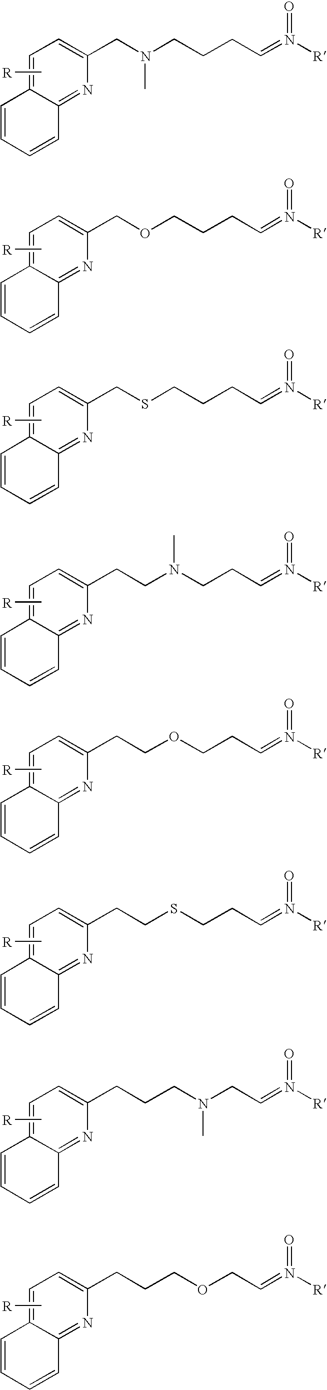 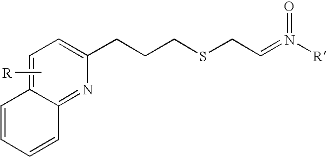
wherein R is R⁴ and R′ is t-butyl, benzyl or cyclohexyl.

73. The pharmaceutical composition of claim 1 wherein the compound is selected from 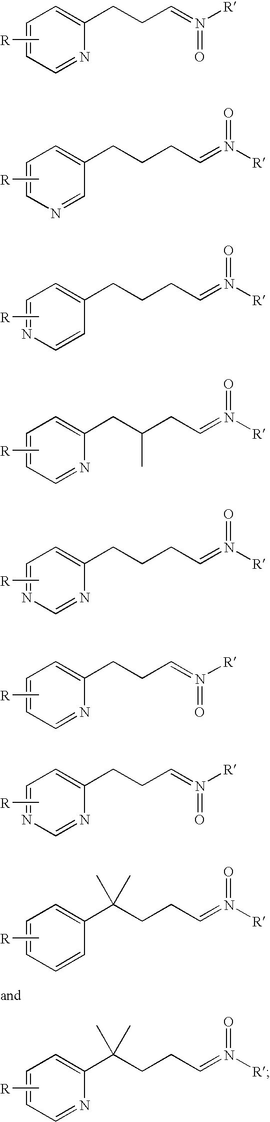
and wherein R is R⁴ and R′ is selected from t-butyl, cyclohexyl or benzyl.

74. (canceled)

75. A pharmaceutical composition comprising a compound selected from
1. Benzenemethanamine, N-[(1S)-2-methyl-1-[(Z)-[oxido(phenylmethyl)imino]methyl]propyl]-N-(phenylmethyl)-
2. Benzeneethanamine, .alpha.-[(Z)-[oxido(phenylmethyl)imino]methyl]-N,N-bis(phenylmethyl)-, (.alpha.S)-
3. Benzenemethanamine, N-[(2S)-2,3-bis(phenylmethoxy)propylidene]-, N-oxide, [N(Z)]-
4. Benzenemethanamine, N-[(2R)-3-fluoro-2-(phenylmethoxy)propylidene]-, N-oxide, [N(Z)]-
5. Benzenemethanamine, N-[(1S)-1-methyl-2-[oxido(phenylmethyl)imino]ethyl]-N-(phenylmethyl)-
6. Glycine, N-[3-(phenylmethoxy)propylidene]-, 1,1-dimethylethyl ester, N-oxide
7. Benzenemethanamine, N-[2-(phenylmethoxy)ethylidene]-, N-oxide,
8. Benzenemethanamine, N-[1-methyl-2-[oxido(phenylmethyl)imino]ethyl]-N-(phenylmethyl)-, [S-(Z)]-
9. Carbamic acid, [2-[oxido(phenylmethyl)imino]-1-(phenylmethyl)ethyl](phenylmethyl)-, 1,1-dimethylethyl ester, [S-(Z)]-
10. Carbamic acid, [1-methyl-2-[oxido(phenylmethyl)imino]ethyl](phenylmethyl)-, 1,1-dimethylethyl ester, [S-(Z)]-
11. Benzenemethanamine, N-[2-(phenylmethoxy)ethylidene]-, N-oxide
12. Benzenemethanamine, N-[2-(phenylmethoxy)propylidene]-, N-oxide, [S-(Z)]-
13. Benzenemethanamine, N-[3-(phenylmethoxy)propylidene]-, N-oxide, (Z)-
14. 2-Butanone, 4-[[1-methyl-2-(methyloxidoimino)ethyl](phenylmethyl)amino]-, (S)-
15. 2-Butanone, 4-[[1-methyl-2-[(phenylmethyl)imino]ethyl](phenylmethyl)amino]-, N-oxide,
16. 2-Butanone, 4-[[2-[(1,1-dimethylethyl)oxidoimino]-1-methylethyl](phenylmethyl)amino]-, (S)-
17. Benzenemethanamine, N-[2-[(1-phenyl-3-butenyl)oxy]ethylidene]-, N-oxide
18. Benzenemethanamine, N-[3-[(4-methoxyphenyl)methoxy]propylidene]-, N-oxide, (Z)-
19. Acetamide, 2-[[4-(dimethylamino)phenyl]imino]-N-2-naphthalenyl-N-phenyl-, N-oxide
20. Acetamide, 2-[[4-(dimethylamino)phenyl]oxidoimino]-N-1-naphthalenyl-N-phenyl-
21. Acetamide, N-1-naphthalenyl-2-(oxidophenylimino)-N-phenyl-
22. 2-Propanol, 1-[(1-methylethyl)imino]-3-(1-naphthalenyloxy)-, N-oxide,
23. Acetamide, 2-[[4-(dimethylamino)phenyl]oxidoimino]-N-(1-methoxy-2-naphthalenyl)-
24. Acetamide, N-1-naphthalenyl-2-(oxidophenylimino)-
25. Methanamine, N-[2-[(4,6-dimethoxy-2-pyrimidinyl)oxy]-3-methylbutylidene]-, N-oxide and
26. Methanamine, N-[2-[(4,6-dimethoxy-2-pyrimidinyl)oxy]-3,3-dimethylbutylidene]-, N-oxide.

76. A unit dosage form of the composition of claim 1 comprising about 10, 25, 50, 100, 500, 1000, 2000 or 2500 mg of the compound according to formula (1).

77. A method of treating or preventing an oxidative condition in a subject in need thereof comprising the step of administering to the subject an effective amount of the composition according to claim 1.

78. A method of treating or preventing an ischemic or ischemia/reperfusion-related condition in a subject in need thereof comprising the step of administering to the subject an effective amount of the composition according to claim 1.

79. The method of claim 77 wherein the subject is a mammal.

80. The method of claim 79 wherein the subject is a human.

81. A kit for treating or preventing an oxidative, ischemic or ischemia/reperfusion mediated condition in a subject in need thereof comprising an effective amount of a compound as recited in claim 1 and a label or labeling with instructions for using the compound to treat or prevent the condition.

82. The method of claim 78 wherein the subject is a mammal.
